(19) United States
(12) Patent Application Publication
(10) Pub. No.: US 2021/0346384 A1
(43) Pub. Date: Nov. 11, 2021

(54) COMBINATIONS OF TGF-BETA INHIBITORS AND CDK INHIBITORS FOR CANCER TREATMENTS

(71) Applicant: PFIZER INC., New York, NY (US)

(72) Inventor: Flavia Mercer PERNASETTI, San Diego, CA (US)

(73) Assignee: PFIZER INC., New York, NY (US)

(21) Appl. No.: 17/277,475

(22) PCT Filed: Sep. 16, 2019
(86) PCT No.: PCT/IB2019/057776

§ 371 Date: Mar. 18, 2021

Related U.S. Application Data
(60) Provisional application No. 62/892,771, filed on Aug. 28, 2019.
(60) Provisional application No. 62/890,168, filed on Aug. 22, 2019.
(60) Provisional application No. 62/733,120, filed on Sep. 19, 2018.
(60) Provisional application No. 62/732,618, filed on Sep. 18, 2018.

(51) Int. Cl. A61K 31/519; A61K 31/565; A61P 35/00; A61K 31/455

(52) U.S. Cl. CPC A61K 31/519; A61K 31/455; A61P 35/00; A61K 31/565

(57) ABSTRACT

This invention relates to a method of treating breast cancer by administering a TGFβ inhibitor in combination with a CDK inhibitor to a patient in need therof.

### FIELD OF THE INVENTION

The present invention relates to combination therapies useful for the treatment of cancers. In particular, this invention relates to methods for treating cancers by administering a TGFβ inhibitor in combination with a CDK inhibitor. Pharmaceutical uses of the combination of the present invention are also described.

### BACKGROUND

TGFβ signaling is an emerging pathway in cancer progression and has a role in modulating immune response, and in many other cancer pathways including metastasis and angiogenesis. Elevated TGFβ expression by tumor and stromal cells in the tumor microenvironment and activation of TGFβ receptor intracellular signaling is observed in many cancers (Massague J. TGFbeta in Cancer. Cell 2008; 134(2):215-30; Neuzillet C, Tijeras-Raballand A, Cohen R, et al. Targeting the TGFß pathway for cancer therapy. Pharmacol Ther 2015; 147:22-31). The TGFβ signaling pathway can be activated upon interaction of dimeric TGFβ ligand with its specific cell-surface transmembrane serine/threonine kinase receptors. The activated TGFβ ligand interacts with TGFβ type II receptors (TGFβR2), which recruit and phosphorylate TGFβ type I receptors (TGFβR1, also known as activin receptor-like kinase (ALK5)) at specific serine and threonine residues (Principe D R, Doll J A, Bauer J, et al. TGF-ß: duality of function between tumor prevention and carcinogenesis. J Natl Cancer Inst 2014; 106(2):djt369). In turn, activated TGFβR1 phosphorylates SMAD2 and SMAD3, which can then assemble into complexes with SMAD4 and translocate to the nucleus, where they regulate the expression of TGFβ target genes (Massague J. TGFbeta in Cancer. Cell 2008; 134(2):215-30). In addition to SMAD signaling, non-SMAD signaling can also be initiated downstream of TGFβ receptors, which can lead to the activation of various pathways such as phosphoinositide 3-kinase (P13K), c-Jun N-terminal kinase (JNK), and extracellular signal-regulated kinase (P38/ERK) mitogen-activated protein (MAP) kinases (Mu Y, Gudey S K, Landström M. Non-Smad signaling pathways. Cell Tissue Res 2012; 347(1):11-20).

Activation of the TGFβ pathway in cancer cells can induce epithelial-to-mesenchymal transition (EMT) in which epithelial cells lose their apico-basal polarity and cell-cell adhesion, to become highly migratory mesenchymal cells, leading to metastasis.

In addition to importance in tumor cell migration and metastasis, EMT has also been linked to tumor cell evasion of immune surveillance (Akalay I, Janji B, Hasmim M, et al. Epithelial-to-mesenchymal transition and autophagy induction in breast carcinoma promote escape from T-cell-mediated lysis. Cancer Res 2013; 73(8):2418-27). TGFβ is a potent immunosuppressive agent on both innate and adaptive immune cells, including dendritic cells, macrophages, natural killer cells, and CD4+ and CD8+ T cells. Conversely, TGFβ has a key role stimulating the differentiation of immune-suppressive regulatory T (Treg) cells and myeloid derived suppressor cells (MDSCs) (Akalay I, Janji B, Hasmim M, et al. Epithelial-to-mesenchymal transition and autophagy induction in breast carcinoma promote escape from T-cell-mediated lysis. Cancer Res 2013; 73(8):2418-27).

TGFβ pathways have key roles in disease progression and resistance to therapy in a broad spectrum of tumors (Neuzillet C, Tijeras-Raballand A, Cohen R, et al. Targeting the TGF11 pathway for cancer therapy. Pharmacol Ther 2015; 147:22-31; Colak S, Ten Dijke P. Targeting TGF-ß signaling in cancer. Trends in Cancer 2017; 3(1):56-71). High TGFβ signatures and EMT gene expression are found in a variety of tumors (Mak M P, Tong P, Diao L, et al. A Patient-Derived, Pan-Cancer EMT Signature Identifies Global Molecular Alterations and Immune Target Enrichment Following Epithelial-to-Mesenchymal Transition. Clin Cancer Res 2016; 22(3):609-20.). TGFβ is an important regulator of the tumor microenvironment by inducing expression of extracellular matrix (ECM) proteins and suppressing expression of chemokines and cytokines required for T cell tumor infiltration, creating a reactive stroma with dense ECM and a T cellexcluded infiltrate phenotype, with peritumoral or stromal T cell localization (Hegde P S, Karanikas V, Evers S. The Where, the When, and the How of Immune Monitoring for Cancer Immunotherapies in the Era of Checkpoint Inhibition. Clin Cancer Res 2016; 22(8):1865-74).

The compound, 4-(2-(5-chloro-2-fluorophenyl)-5-isopropylpyridin-4-ylamino)-N-(1,3-dihydroxypropan-2-Anicotinamide (also referred to as “PF-06952229” or “PF-‘2229”), is a potent and selective TGFβ (transforming growth factor beta) inhibitor, having the structure:

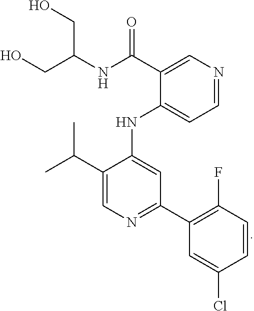 [SMILES: CC(C)c1cnc(-c2cc(Cl)ccc2F)cc1Nc1ccncc1C(=O)NC(CO)CO]

PF-06952229 and pharmaceutically acceptable salts thereof are disclosed in International Publication No. W02015/103355 and U.S. Pat. No. 10,030,004. The contents of each of the foregoing references are incorporated herein by reference in their entirety.

Cyclin-dependent kinases (CDKs) are important cellular enzymes that perform essential functions in regulating eukaryotic cell division and proliferation. The cyclin-dependent kinase catalytic units are activated by regulatory subunits known as cyclins. At least sixteen mammalian cyclins have been identified (Johnson D G, Walker C L. Cyclins and Cell Cycle Checkpoints. Annu. Rev. Pharmacol. Toxicol. (1999) 39:295-312). Cyclin B/CDK1, cyclin A/CDK2, cyclin E/CDK2, cyclin D/CDK4, cyclin D/CDK6, and likely other heterodynes are important regulators of cell cycle progression. Additional functions of cyclin/CDK heterodynes include regulation of transcription, DNA repair, differentiation and apoptosis (Morgan DO. Cyclin-dependent kinases: engines, clocks, and microprocessors. Annu. Rev. Cell. Dev. Biol. (1997) 13:261-291).

Cyclin-dependent kinase inhibitors have been demonstrated to be useful in treating cancer. Increased activity or temporally abnormal activation of cyclin-dependent kinases has been shown to result in the development of human tumors, and human tumor development is commonly associated with alterations in either the CDK proteins themselves or their regulators (Cordon-Cardo C. Mutations of cell cycle regulators: biological and clinical implications for human neoplasia. Am. J. Pathol. (1995) 147:545-560; Karp J E, Broder S. Molecular foundations of cancer: new targets for intervention. Nat. Med. (1995) 1:309-320; Hall M, Peters G. Genetic alterations of cyclins, cyclin-dependent kinases, and Cdk inhibitors in human cancer. Adv. Cancer Res. (1996) 68:67-108). Amplifications of the regulatory subunits of CDKs and cyclins, and mutation, gene deletion, or transcriptional silencing of endogenous CDK inhibitors have also been reported (Smalley et al. Identification of a novel subgroup of melanomas with KIT/cyclin-dependent kinase-4 overexpression. Cancer Res (2008) 68: 5743-52).

Clinical trials for the CDK4/6 inhibitors palbociclib, ribociclib and abemaciclib are ongoing for breast and other cancers, as single agents or in combination with other therapeutics. Palbociclib, ribociclib and abemaciclib have been approved for treatment of hormone receptor (HR)-positive, human epidermal growth factor receptor 2 (HER2)-negative advanced or metastatic breast cancer in combination with aromatase inhibitors, such as letrozole, in a first line setting and with fulvestrant in second or later lines of therapy in certain patients. (O'Leary et al. Treating cancer with selective CDK4/6 inhbitors. Nature Reviews (2016) 13:417-430). While CDK4/6 inhibitors have shown significant clinical efficacy in ER-positive metastatic breast cancer, as with other kinases their effects may be limited over time by the development of primary or acquired resistance.

Overexpression of CDK2 is associated with abnormal regulation of cell-cycle. The cyclin E/CDK2 complex plays and important role in regulation of the G1/S transition, histone biosynthesis and centrosome duplication. Progressive phosphorylation of Rb by cyclin D/Cdk4/6 and cyclin E/Cdk2 releases the G1 transcription factor, E2F, and promotes S-phase entry. Activation of cyclin A/CDK2 during early S-phase promotes phosphorylation of endogenous substrates that permit DNA replication and inactivation of E2F, for S-phase completion. (Asghar et al. The history and future of targeting cyclin-dependent kinases in cancer therapy, Nat. Rev. Drug. Discov. 2015; 14(2): 130-146). Cyclin E, the regulatory cyclin for CDK2, is frequently overexpressed in cancer. Cyclin E amplification or overexpression has long been associated with poor outcomes in breast cancer. (Keyomarsi et al., Cyclin E and survival in patients with breast cancer. N Engl J Med. (2002) 347:1566-75). Cyclin E2 (CCNE2) overexpression is associated with endocrine resistance in breast cancer cells and CDK2 inhibition has been reported to restore sensitivity to tamoxifen or CDK4 inhibitors in tamoxifen-resistant and CCNE2 overexpressing cells. (Caldon et al., Cyclin E2 overexpression is associated with endocrine resistance but not insensitivity to CDK2 inhibition in human breast cancer cells. Mol. Cancer Ther. (2012) 11:1488-99; Herrera-Abreu et al., Early Adaptation and Acquired Resistance to CDK4/6 Inhibition in Estrogen Receptor-Positive Breast Cancer, Cancer Res. (2016) 76: 2301-2313). Cyclin E amplification also reportedly contributes to trastuzumab resistance in HER2+ breast cancer. (Scaltriti et al. Cyclin E amplification/overexpression is a mechanism of trastuzumab resistance in HER2+ breast cancer patients, Proc Natl Acad Sci. (2011) 108: 3761-6). Cyclin E overexpression has also been reported to play a role in basal-like and triple negative breast cancer (TNBC), as well as inflammatory breast cancer. (Elsawaf & Sinn, Triple Negative Breast Cancer: Clinical and Histological Correlations, Breast Care (2011) 6:273-278; Alexander et al., Cyclin E overexpression as a biomarker for combination treatment strategies in inflammatory breast cancer, Oncotarget (2017) 8: 14897-14911.)

Palbociclib, or 6-acetyl-8-cyclopentyl-5-methyl-2-(5-piperazin-1-yl-pyridin-2-ylamino)-8H-pyrido[2,3-d]pyrimidin-7-one (also referred to as PD-0332991) is a potent and selective inhibitor of CDK4 and CDK6, having the structure:

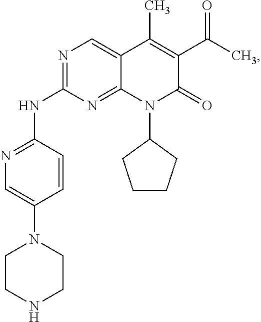 [SMILES: CC(=O)c1c(C)c2cnc(Nc3ccc(N4CCNCC4)cn3)nc2n(C2CCCC2)c1=O]

Palbociclib is described in WHO Drug Information, Vol. 27, No. 2, page 172 (2013). Palbociclib and pharmaceutically acceptable salts thereof are disclosed in International Publication No. WO 2003/062236 and U.S. Pat. Nos. 6,936,612, 7,208,489 and 7,456,168; International Publication No. WO 2005/005426 and U.S. Pat. Nos. 7,345,171 and 7,863,278; International Publication No. WO 2008/032157 and U.S. Pat. No. 7,781,583; and International Publication No. WO 2014/128588. The contents of each of the foregoing references are incorporated herein by reference in their entirety.

PF-06873600, or 6-(difluoromethyl)-84(1R,2R)-2-hydroxy-2-methylcyclopentyl)-2-(1-(methylsulfonyl)piperidin-4-ylamino)pyrido[2,3-d]pyrimidin-7(8H)-one, is a potent and selective inhibitor of CDK2, CDK4 and CDK6, having the structure:

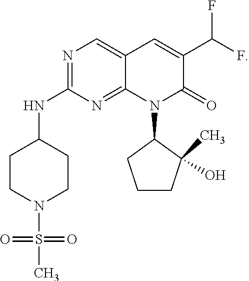 [SMILES: C[C@@]1(O)CCC[C@H]1n1c(=O)c(C(F)F)cc2cnc(NC3CCN(S(C)(=O)=O)CC3)nc21]

PF-06873600 is disclosed in International Publication No. WO 2018/033815 published Feb. 22, 2018. The contents of that reference are incorporated herein by reference in their entirety.

While the selective CDK4/6 inhibitor palbociclib has proven to be clinically efficacious in breast cancer (DeMichele A, Clark A S, Tan K S, et al. CDK 4/6 inhibitor palbociclib (PD-0332991) in Rb+advanced breast cancer: phase II activity, safety, and predictive biomarker assessment. Clin Cancer Res 2015; 21(5):995-1001; Finn R S, Martin M, Rugo H S, et al. Palbociclib and Letrozole in Advanced Breast Cancer. New Engl J Med 2016; 375(20):1925-36; Cristofanilli M, Turner N C, Bondarenko I, et al. Fulvestrant plus palbociclib versus fulvestrant plus placebo for treatment of hormone-receptor-positive, HER2-negative metastatic breast cancer that progressed on previous endocrine therapy (PALOMA-3): final analysis of the multicentre, double-blind, phase 3 randomised controlled trial. Lancet Oncol 2016; 17(4):425-39), after initial clinical benefit, acquired resistance to palbociclib may occur (Knudsen Erik S., Witkiewicz Agnieszka K., The Strange Case of CDK4/6 Inhibitors: Mechanisms, Resistance, and Combination Strategies. Trends Cancer 2017; 3(1):39-55). In preclinical studies, treatment of tumor cells with palbociclib induces TGFβ and EMT gene signature expression, enhancing tumor cell invasiveness.

Improved combination therapies for the treatment of breast cancers, including breast cancers resistant to CDK inhibitors, comprise a large unmet medical need and the identification of novel combination regimens are required to improve treatment outcome.

### SUMMARY OF THE INVENTION

Each of the embodiments described below can be combined with any other embodiment described herein not inconsistent with the embodiment with which it is combined. Furthermore, each of the embodiments described herein envisions within its scope pharmaceutically acceptable salts of the compounds described herein. Accordingly, the phrase “or a pharmaceutically acceptable salt thereof” is implicit in the description of all compounds described herein.

Embodiments described herein relate to a method for treating breast cancer, particularly HR-positive, HER2-negative advanced or metastatic breast cancer, comprising administering to a patient in need thereof an amount of a TGFβ inhibitor and an amount of a CDK inhibitor, wherein the amounts together are effective in treating said cancer. Further aspects of this embodiment include administration of a third component which is an aromatase inhibitor or fulvestrant.

Additional embodiments described herein relate to a method for treating breast cancer, particularly HR-positive, HER2-negative advanced or metastatic breast cancer, comprising administering to a patient in need thereof a synergistic amount of a TGFβ inhibitor in combination with a CDK inhibitor. Further aspects of this embodiment include administration of a third component which is an aromatase inhibitor or fulvestrant.

Further embodiments described herein relate to a combination of a TGFβ inhibitor inhibitor and a CDK inhibitor for use in the treatment of breast cancer, particularly HR-positive, HER2-negative advanced or metastatic breast cancer. Further aspects of this embodiment include administration of a third component which is an aromatase inhibitor or fulvestrant.

Some embodiments described herein relate to a use of a TGFβ inhibitor and a CDK inhibitor, in the manufacture of a medicament for the treatment of breast cancer, particularly HR-positive, HER2-negative advanced or metastatic breast cancer. Further aspects of this embodiment include use of a third component which is an aromatase inhibitor or fulvestrant.

Additional embodiments described herein relate to a combination of a TGFβ inhibitor and a CDK inhibitor for use in the treatment of breast cancer, particularly HR-positive, HER2-negative advanced or metastatic breast cancer, wherein the combination is synergistic. Further aspects of this embodiment include combinations that also include a third component which is an aromatase inhibitor or fulvestrant.

Some embodiments described herein relate to a use of a synergistic amount of a TGFβ inhibitor and a CDK inhibitor, in the manufacture of a medicament for the treatment of breast cancer, particularly HR-positive, HER2-negative advanced or metastatic breast cancer. Further aspects of this embodiment include use of a third component which is an aromatase inhibitor or fulvestrant.

In certain embodiments of the method or use of the present invention, the TGFβ inhibitor is selected from the group consisting of galunisertib, LY2109761, SB525334, SP505124, GW788388, LY364947, RepSox, SD-208, vactosertib, LY3200882 and 4-(2-(5-chloro-2-fluorophenyl)-5-isopropylpyridin-4-ylamino)-N-(1,3-dihydroxypropan-2-yl)nicotinamide (PF-06952229), or a pharmaceutically acceptable salt thereof.

In certain embodiments of the method or use of the present invention, the TGFβ inhibitor is 4-(2-(5-chloro-2-fluorophenyl)-5-isopropylpyridin-4-ylamino)-N-(1,3-dihydroxypropan-2-yl)nicotinamide (PF-06952229), or a pharmaceutically acceptable salt thereof.

In certain embodiments of the method or use of the present invention, the CDK inhibitor is a CDK 4/6 inhibitor or is a CDK 2/4/6 inhibitor.

In certain embodiments of the method or use of the present invention, the CDK inhibitor is a CDK 4/6 inhibitor.

In some embodiments of the method or use of the present invention, the CDK 4/6 inhibitor is selected from the group consisting of abemaciclib, ribociclib and palbociclib, or a pharmaceutically acceptable salt thereof.

In some embodiments of the method or use of the present invention, the CDK 4/6 inhibitor is palbociclib, or a pharmaceutically acceptable salt thereof.

In certain embodiments of the method or use of the present invention, the CDK inhibitor is a CDK 2/4/6 inhibitor.

In some embodiments of the method or use of the present invention, the CDK 2/4/6 inhibitor is 6-(difluoromethyl)-8-((1R,2R)-2-hydroxy-2-methylcyclopentyl)-2-(1-(methylsulfonyl)piperidin-4-ylami no)pyrido[2 ,3-d]pyri midin-7(8H)-one (“PF-06873600”), or a pharmaceutically acceptable salt thereof.

Embodiments described herein relate to a method for treating breast cancer, particularly HR-positive, HER2-negative advanced or metastatic breast cancer, comprising administering to a patient in need thereof an amount of 4-(2-(5-chloro-2-fluorophenyl)-5-isopropylpyridin-4-ylamino)-N-(1,3-dihydroxypropan-2-yl)nicotinamide (PF-06952229), or a pharmaceutically acceptable salt thereof, and an amount of palbociclib, or a pharmaceutically acceptable salt thereof, wherein the amounts together are effective in treating breast cancer.

Additional embodiments described herein relate to a method for treating breast cancer, particularly HR-positive, HER2-negative advanced or metastatic breast cancer, comprising administering to a patient in need thereof a synergistic amount of 4-(2-(5-chloro-2-fluorophenyl)-5-isopropylpyridin-4-ylamino)- N-(1, 3-di hydroxypropan-2-yl)nicotinamide (PF-06952229), or a pharmaceutically acceptable salt thereof, in combination with palbociclib, or a pharmaceutically acceptable salt thereof.

Further embodiments described herein relate to a combination of 4-(2-(5-chloro-2-fluorophenyl)-5-isopropylpyridin-4-ylamino)-N-(1,3-dihydroxypropan-2-yl)nicotinamide (PF-06952229), or a pharmaceutically acceptable salt thereof, and palbociclib, or a pharmaceutically acceptable salt thereof, for use in the treatment of breast cancer, particularly HR-positive, HER2-negative advanced or metastatic breast cancer.

Some embodiments described herein relate to a use of 4-(2-(5-chloro-2-fluorophenyl)-5-isopropylpyridin-4-ylamino)-N-(1,3-dihydroxypropan-2-yl)nicotinamide (PF-06952229), or a pharmaceutically acceptable salt thereof, and palbociclib, or a pharmaceutically acceptable salt thereof, in the manufacture of a medicament for the treatment of breast cancer, particularly HR-positive, HER2-negative advanced or metastatic breast cancer.

Additional embodiments described herein relate to a combination of 4-(2-(5-chloro-2-fluorophenyl)-5-isopropylpyridin-4-ylamino)-N-(1,3-dihydroxypropan-2-Anicotinamide (PF-06952229), or a pharmaceutically acceptable salt thereof, and palbociclib, or a pharmaceutically acceptable salt thereof, for use in the treatment of breast cancer, particularly HR-positive, HER2-negative advanced or metastatic breast cancer, wherein the combination is synergistic.

Some embodiments described herein relate to a use of a synergistic amount of 4-(2-(5-chloro-2-fluorophenyl)-5-isopropylpyridin-4-ylamino)-N-(1,3-dihydroxypropan-2-yl)nicotinamide (PF-06952229), or a pharmaceutically acceptable salt thereof, and palbociclib, or a pharmaceutically acceptable salt thereof, in the manufacture of a medicament for the treatment of breast cancer, particularly HR-positive, HER2-negative advanced or metastatic breast cancer.

Further embodiments described herein relate to a combination of a TGFβ inhibitor and a CDK inhibitor, for use in the treatment of breast cancer, particularly HR-positive, HER2-negative advanced or metastatic breast cancer, wherein the CDK inhibitor is administered according to a non-standard clinical dosing regimen.

Additional embodiments described herein relate to a use of a TGFβ inhibitor and a CDK inhibitor, in the manufacture of a medicament for the treatment of breast cancer, particularly HR-positive, HER2-negative advanced or metastatic breast cancer, wherein the CDK inhibitor is administered according to a non-standard clinical dosing regimen.

In embodiments of the method or use of the present invention, the non-standard clinical dosing regimen is a non-standard clinical dose.

In embodiments of the method or use of the present invention, the non-standard clinical dose is a low-dose amount of the CDK inhibitor.

In embodiments of the method or use of the present invention, the non-standard clinical dosing regimen is a non-standard dosing schedule.

In embodiments of the method or use of the present invention, the non-standard dosing schedule is a continuous dosing schedule of the CDK inhibitor.

In embodiments of the method or use of the present invention, the CDK inhibitor is a CDK 4/6 inhibitor.

In embodiments of the method or use of the present invention, the TGFβ inhibitor is 4-(2-(5-chloro-2-fluorophenyl)-5-isopropylpyridin-4-ylamino)-N-(1,3-dihydroxypropan-2-yl)nicotinamide (PF-06952229), or a pharmaceutically acceptable salt thereof, and the CDK inhibitor is palbociclib, or a pharmaceutically acceptable salt thereof.

Embodiments described herein relate to a method for treating breast cancer, particularly HR-positive, HER2-negative advanced or metastatic breast cancer, comprising administering to a patient in need thereof an amount of 4-(2-(5-chloro-2-fluorophenyl)-5-isopropylpyridin-4-ylamino)-N-(1,3-dihydroxypropan-2-yl)nicotinamide (PF-06952229), or a pharmaceutically acceptable salt thereof, and an amount of palbociclib, or a pharmaceutically acceptable salt thereof, wherein palbociclib, or a pharmaceutically acceptable salt thereof, is administered according to a non-standard clinical dosing regimen, and further wherein the amounts together are effective in treating breast cancer, particularly HR-positive, HER2-negative advanced or metastatic breast cancer.

Further embodiments described herein relate to a combination of 4-(2-(5-chloro-2-fluorophenyl)-5-isopropylpyridin-4-ylamino)-N-(1,3-dihydroxypropan-2-yl)nicotinamide (“PF-06952229”), or a pharmaceutically acceptable salt thereof, and palbociclib, or a pharmaceutically acceptable salt thereof, for use in the treatment of breast cancer, particularly HR-positive, HER2-negative advanced or metastatic breast cancer, wherein palbociclib, or a pharmaceutically acceptable salt thereof, is administered according to a non-standard clinical dosing regimen.

Additional embodiments described herein relate to a use of 4-(2-(5-chloro-2-fluorophenyl)-5-isopropylpyridin-4-ylamino)-N-(1,3-dihydroxypropan-2-yl)nicotinamide (PF-06952229), or a pharmaceutically acceptable salt thereof, and palbociclib, or a pharmaceutically acceptable salt thereof, in the manufacture of a medicament for the treatment of breast cancer, particularly HR-positive, HER2-negative advanced or metastatic breast cancer, wherein palbociclib, or a pharmaceutically acceptable salt thereof, is administered according to a non-standard clinical dosing regimen.

In embodiments of the method or use of the present invention, the non-standard clinical dosing regimen is a non-standard clinical dose.

In embodiments of the method or use of the present invention, the non-standard clinical dose is a low-dose amount of palbociclib, or a pharmaceutically acceptable salt thereof.

In embodiments of the method or use of the present invention, the low-dose amount of palbociclib, or a pharmaceutically acceptable salt thereof, is about 50 mg, about 75 mg or about 100 mg once daily.

In embodiments of the method or use of the present invention, the low-dose amount of palbociclib, or a pharmaceutically acceptable salt thereof, is about 75 mg once daily.

In embodiments of the method or use of the present invention, the low-dose amount of palbociclib, or a pharmaceutically acceptable salt thereof, is about 100 mg once daily.

In embodiments of the method or use of the present invention, the non-standard clinical dosing regimen is a non-standard dosing schedule.

In embodiments of the method or use of the present invention, the non-standard dosing schedule is a continuous dosing schedule of palbociclib, or a pharmaceutically acceptable salt thereof.

In embodiments of the method or use of the present invention, the continuous dosing schedule of palbociclib, or a pharmaceutically acceptable salt thereof, is a complete cycle of 21 days.

In embodiments of the method or use of the present invention, the continuous dosing schedule of palbociclib, or a pharmaceutically acceptable salt thereof, is a complete cycle of 28 days.

In embodiments of the method or use of the present invention, the non-standard dosing schedule comprises administering palbociclib, or a pharmaceutically acceptable salt thereof, once daily for 14 consecutive days followed by 7 days off treatment.

In embodiments of the method or use of the present invention, the non-standard clinical dosing regimen comprises administering about 75 mg of palbociclib, or a pharmaceutically acceptable salt thereof, once daily for 14 consecutive days followed by 7 days off treatment.

Embodiments described herein relate to a method for treating breast cancer, particularly HR-positive, HER2-negative advanced or metastatic breast cancer, comprising administering to a patient in need thereof an amount of 4-(2-(5-chloro-2-fluorophenyl)-5-isopropylpyridin-4-ylamino)-N-(1,3-dihydroxypropan-2-yl)nicotinamide (PF-06952229), or a pharmaceutically acceptable salt thereof, and an amount of palbociclib, or a pharmaceutically acceptable salt thereof, wherein palbociclib, or a pharmaceutically acceptable salt thereof, is administered according to a non-standard clinical dosing regimen, and further wherein the amounts together are effective in treating breast cancer, particularly HR-positive, HER2-negative advanced or metastatic breast cancer.

Further embodiments described herein relate to a combination of 4-(2-(5-chloro-2-fluorophenyl)-5-isopropylpyridin-4-ylamino)-N-(1,3-dihydroxypropan-2-yl)nicotinamide (PF-06952229), or a pharmaceutically acceptable salt thereof, and palbociclib, or a pharmaceutically acceptable salt thereof, for use in the treatment of breast cancer, particularly HR-positive, HER2-negative advanced or metastatic breast cancer, wherein palbociclib, or a pharmaceutically acceptable salt thereof, is administered according to a non-standard clinical dosing regimen.

Additional embodiments described herein relate to a use of 4-(2-(5-chloro-2-fluorophenyl)-5-isopropylpyridin-4-ylamino)-N-(1,3-dihydroxypropan-2-yl)nicotinamide (PF-06952229), or a pharmaceutically acceptable salt thereof, and palbociclib, or a pharmaceutically acceptable salt thereof, in the manufacture of a medicament for the treatment of breast cancer, particularly HR-positive, HER2-negative advanced or metastatic breast cancer, wherein palbociclib, or a pharmaceutically acceptable salt thereof, is administered according to a non-standard clinical dosing regimen.

Embodiments described herein relate to a synergistic combination of

(a) A TGFβ inhibitor; and

(b) a CDK inhibitor.

Further embodiments described herein relate to a synergistic combination of

(a) an TGFβ inhibitor; and

(b) a CDK inhibitor,

wherein component (a) and component (b) are synergistic.

Additional embodiments, relate to a pharmaceutical composition of a TGFβ inhibitor and a pharmaceutical composition of a CDK inhibitor for use in the treatment of breast cancer, particularly HR-positive, HER2-negative advanced or metastatic breast cancer.

In embodiments of combination of the present invention, the TGFβ is 4-(2-(5-chloro-2-fluorophenyl)-5-isopropylpyridin-4-ylamino)- N-(1, 3-di hydroxypropan-2-yl)nicotinamide (PF-06952229), or a pharmaceutically acceptable salt thereof.

In embodiments of combination of the present invention, the CDK inhibitor is a CDK 4/6 inhibitor.

In embodiments of combination of the present invention, the CDK 4/6 inhibitor is selected from the group consisting of abemaciclib, ribociclib and palbociclib, or a pharmaceutically acceptable salt thereof.

In embodiments of combination of the present invention, the CDK 4/6 inhibitor is palbociclib, or a pharmaceutically acceptable salt thereof.

In embodiments of combination of the present invention, the TGFβ inhibitor is 4-(2-(5-chloro-2-fluorophenyl)-5-isopropylpyridin-4-ylamino)-N-(1,3-dihydroxypropan-2-yl)nicotinamide (PF-06952229), or a pharmaceutically acceptable salt thereof, and the CDK inhibitor is palbociclib, or a pharmaceutically acceptable salt thereof.

### BRIEF DESCRIPTION OF THE DRAWINGS

FIG. 1 shows survival curves of CT26 tumor bearing mice treated with vehicle, PF-0332991, PF-06873600, PF-06952229, the combination of PF-06952229 and PD-0332991, or the combination of PF-06952229 and PF-06873600.

FIG. 2 shows tumor volume in the CT26 Syngenic Tumor Model at day 17 post treatment, for vehicle, PF-06952229, PF-0332991, PF-06783600, the combination of PF-06952229 and PD-0332991, or the combination of PF-06952229 and PF-06783600. These combinations are shown to increase tumor growth inhibition.

FIG. 3 shows tumor volume in the MCF-7 ER⁺ Breast Cancer Tumor Model at day 21 post treatment, for vehicle, PF-06952229, PF-0332991, and the combination of PF-06952229 and PD-0332991. These combinations are shown to increase tumor growth inhibition.

FIG. 4 shows tumor volume in the MCF-7 ER⁺ Breast Cancer Tumor Model at day 21 post treatment, for vehicle, PF-06952229, the combination of PF-0332991 and fulvestrant, and the combination of PF-06952229 and PD-0332991 and fulvestrant. These combinations are shown to increase tumor growth inhibition.

FIG. 5 shows the addition of TGFβ inhibitor PF-06952229 treatment to mice previously receiving CDK4/6 Inhibitor Palbociclib or Palbociclib +Fulvestrant for 21 Days and shows a trend towards increased tumor growth inhibition in the MCF7 ER⁺ xenograft breast cancer tumor model on day 66 post-treatment initiation.

FIG. 6 shows the combination of TGFβ inhibitor PF-06952229 with CDK4/6 inhibitor palbociclib (PD-0332991) or palbociclib+fulvestrant for 21 days results in improved inhibition of pSMAD2 in the MCF7 ER⁺ xenograft breast cancer tumor model.

FIG. 7 shows the combination of TGFβ inhibitor PF-06952229 with CDK4/6 inhibitor palbociclib (PD-0332991)+fulvestrant for 21 days results in improved inhibition of pS807/811 Rb in the MCF7 ER⁺ xenograft breast cancer tumor model.

### DETAILED DESCRIPTION OF THE INVENTION

The present invention may be understood more readily by reference to the following detailed description of the preferred embodiments of the invention and the Examples included herein. It is to be understood that the terminology used herein is for the purpose of describing specific embodiments only and is not intended to be limiting. It is further to be understood that unless specifically defined herein, the terminology used herein is to be given its traditional meaning as known in the relevant art.

As used herein, the singular form “a”, “an”, and “the” include plural references unless indicated otherwise. For example, “an” excipient includes one or more excipients.

As used herein, the term “about” when used to modify a numerically defined parameter (e.g., the dose of a TGFβ inhibitor or a CDK inhibitor) means that the parameter may vary by as much as 10% below or above the stated numerical value for that parameter. For example, a dose of about 5 mg may vary between 4.5 mg and 5.5 mg.

As used herein, terms, including, but not limited to, “agent”, “component”, “composition, “compound”, “substance”, “targeted agent”, “targeted therapeutic agent”, and “therapeutic agent” may be used interchangeably to refer to the compounds of the present invention, specifically a TGFβ inhibitor and a CDK inhibitor.

The following abbreviations may be used herein: DMSO (dimethylsulphoxide); FBS (fetal bovine serum); RPMI (Roswell Park Memorial Institute); mpk (mg/kg or mg drug per kg body weight of animal); and w/w (weight per weight).

Cyclin-dependent kinases (CDKs) and related serine/threonine kinases are important cellular enzymes that perform essential functions in regulating cell division and proliferation. CDK inhibitors include Pan-CDK inhibitors that target a broad spectrum of CDKs or selective CDK inhibitors that target specific CDK(s). CDK inhibitors may have activity against targets in addition to CDKs, such as Aurora A, Aurora B, Chk1, Chk2, ERK1, ERK2, GST-ERK1, GSK-3α, GSK-3β, PDGFR, TrkA and VEGFR. CDK inhibitors include, but are not limited to, abemaciclib, alvocidib, dinaciclib, palbociclib, ribociclib, trilaciclib, lerociclib, roscovitine, AT7519, AZD5438, BMS-265246, BMS-387032, BS-181, JNJ-7706621, K03861, MK-8776, P276-00, PHA-793887, R547, RO-3306 and SU 9516. Examples of Pan-CDK inhibitors include, but are not limited to, alvocidib, dinaciclib, roscovitine, AT7519, AZD5438, BMS-387032, P276-00, PHA-793887, R547 and SU 9516. A non-limiting example of a CDK1 inhibitor is RO-3306. Examples of CDK2 inhibitors include, but are not limited to, K03861 and MK-8776. Examples of CDK1/2 inhibitors include, but are not limited to, BMS-265246 and JNJ-7706621. Examples of CDK4/6 inhibitors include, but are not limited to, abemaciclib, ribociclib and palbociclib. A non-limiting example of a CDK7 inhibitor is BS-181.

In an embodiment, CDK4/6 inhibitors of the present invention include palbociclib. Unless otherwise indicated herein, palbociclib (also referred to herein as “palbo” or “Palbo”) refers to 6-acetyl-8-cyclopentyl-5-methyl-2-(5-piperazin-1-yl-pyridin-2-ylamino)-8H-pyrido[2,3-d]pyrimidin-7-one, or a pharmaceutically acceptable salt thereof.

Some embodiments relate to the pharmaceutically acceptable salts of the compounds described herein. Pharmaceutically acceptable salts of the compounds described herein include the acid addition and base addition salts thereof.

Some embodiments also relate to the pharmaceutically acceptable acid addition salts of the compounds described herein. Suitable acid addition salts are formed from acids which form non-toxic salts. Non-limiting examples of suitable acid addition salts, i.e., salts containing pharmacologically acceptable anions, include, but are not limited to, the acetate, acid citrate, adipate, aspartate, benzoate, besylate, bicarbonate/carbonate, bisulphate/sulphate, bitartrate,borate, camsylate, citrate, cyclamate, edisylate, esylate, ethanesulfonate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methanesulfonate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, p-toluenesulfonate, tosylate, trifluoroacetate and xinofoate salts.

Additional embodiments relate to base addition salts of the compounds described herein. Suitable base addition salts are formed from bases which form non-toxic salts. Non-limiting examples of suitable base salts include the aluminum, arginine, benzathine, calcium, choline, diethylamine, diolamine, glycine, lysine, magnesium, meglumine, olamine, potassium, sodium, tromethamine and zinc salts.

The compounds described herein that are basic in nature are capable of forming a wide variety of salts with various inorganic and organic acids. The acids that may be used to prepare pharmaceutically acceptable acid addition salts of such basic compounds described herein are those that form non-toxic acid addition salts, e.g., salts containing pharmacologically acceptable anions, such as the hydrochloride, hydrobromide, hydroiodide, nitrate, sulfate, bisulfate, phosphate, acid phosphate, isonicotinate, acetate, lactate, salicylate, citrate, acid citrate, tartrate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate and pamoate [i.e., 1,1′-methylene-bis-(2-hydroxy-3-naphthoate)] salts.

The compounds described herein that include a basic moiety, such as an amino group, may form pharmaceutically acceptable salts with various amino acids, in addition to the acids mentioned above.

The chemical bases that may be used as reagents to prepare pharmaceutically acceptable base salts of those compounds of the compounds described herein that are acidic in nature are those that form non-toxic base salts with such compounds. Such non-toxic base salts include, but are not limited to those derived from such pharmacologically acceptable cations such as alkali metal cations (e.g., potassium and sodium) and alkaline earth metal cations (e.g., calcium and magnesium), ammonium or water-soluble amine addition salts such as N-methylglucamine-(meglumine), and the lower alkanolammonium and other base salts of pharmaceutically acceptable organic amines.

Hemisalts of acids and bases may also be formed, for example, hemisulphate and hemicalcium salts.

For a review on suitable salts, see Handbook of Pharmaceutical Salts: Properties, Selection, and Use by Stahl and Wermuth (Wiley-VCH, 2002). Methods for making pharmaceutically acceptable salts of compounds described herein are known to one of skill in the art.

The term “solvate” is used herein to describe a molecular complex comprising a compound described herein and one or more pharmaceutically acceptable solvent molecules, for example, water and ethanol.

The compounds described herein may also exist in unsolvated and solvated forms. Accordingly, some embodiments relate to the hydrates and solvates of the compounds described herein.

Compounds described herein containing one or more asymmetric carbon atoms can exist as two or more stereoisomers. Where a compound described herein contains an alkenyl or alkenylene group, geometric cis/trans (or Z/E) isomers are possible. Where structural isomers are interconvertible via a low energy barrier, tautomeric isomerism (‘tautomerism’) can occur. This can take the form of proton tautomerism in compounds described herein containing, for example, an imino, keto, or oxime group, or so-called valence tautomerism in compounds which contain an aromatic moiety. A single compound may exhibit more than one type of isomerism.

The compounds of the embodiments described herein include all stereoisomers (e.g., cis and trans isomers) and all optical isomers of compounds described herein (e.g., R and S enantiomers), as well as racemic, diastereomeric and other mixtures of such isomers. While all stereoisomers are encompassed within the scope of our claims, one skilled in the art will recognize that particular stereoisomers may be preferred.

In some embodiments, the compounds described herein can exist in several tautomeric forms, including the enol and imine form, and the keto and enamine form and geometric isomers and mixtures thereof. All such tautomeric forms are included within the scope of the present embodiments. Tautomers exist as mixtures of a tautomeric set in solution. In solid form, usually one tautomer predominates. Even though one tautomer may be described, the present embodiments include all tautomers of the present compounds.

Included within the scope of the present embodiments are all stereoisomers, geometric isomers and tautomeric forms of the compounds described herein, including compounds exhibiting more than one type of isomerism, and mixtures of one or more thereof. Also included are acid addition or base salts wherein the counterion is optically active, for example, d-lactate or 1-lysine, or racemic, for example, dl-tartrate or dl-arginine.

The present embodiments also include atropisomers of the compounds described herein. Atropisomers refer to compounds that can be separated into rotationally restricted isomers.

Cis/trans isomers may be separated by conventional techniques well known to those skilled in the art, for example, chromatography and fractional crystallization.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where a compound described herein contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person.

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above.

A “patient” to be treated according to this invention includes any warm-blooded animal, such as, but not limited to human, monkey or other lower-order primate, horse, dog, rabbit, guinea pig, or mouse. For example, the patient is human. Those skilled in the medical art are readily able to identify individual patients who are afflicted with breast cancer, particularly HR-positive, HER2-negative advanced or metastatic breast cancer and who are in need of treatment.

The term “advanced”, as used herein, as it relates to breast cancer, includes locally advanced (non-metastatic) disease and metastic disease. Locally advanced breast , which may or may not be be treated with curative intent, and metastatic disease, which cannot be treated with curative intent are included within the scope of “advanced breast cancer, as used in the present invention. Those skilled in the art will be able to recognize and diagnose advanced breast cancer in a patient.

“Duration of Response” for purposes of the present invention means the time from documentation of tumor model growth inhibition due to drug treatment to the time of acquisition of a restored growth rate similar to pretreatment growth rate.

The term “additive” is used to mean that the result of the combination of two compounds, components or targeted agents is no greater that the sum of each compound, component or targeted agent individually. The term “additive” means that there is no improvement in the disease condition or disorder being treated over the use of each compound, component or targeted agent individually.

The terms “synergy” or “synergistic” are used to mean that the result of the combination of two compounds, components or targeted agents is greater than the sum of each agent together. The terms “synergy” or “synergistic” means that there is an improvement in the disease condition or disorder being treated, over the use of each compound, component or targeted agent individually. This improvement in the disease condition or disorder being treated is a “synergistic effect”. A “synergistic amount” is an amount of the combination of the two compounds, components or targeted agents that results in a synergistic effect, as “synergistic” is defined herein.

Determining a synergistic interaction between one or two components, the optimum range for the effect and absolute dose ranges of each component for the effect may be definitively measured by administration of the components over different w/w ratio ranges and doses to patients in need of treatment. However, the observation of synergy in in vitro models or in vivo models can be predictive of the effect in humans and other species and in vitro models or in vivo models exist, as described herein, to measure a synergistic effect and the results of such studies can also be used to predict effective dose and plasma concentration ratio ranges and the absolute doses and plasma concertrations required in humans and other species by the application of pharmacokinetic/pharmacodynamic methods.

In accordance with the present invention, an amount of a first compound or component is combined with an amount of a second compound or component, and the amounts together are effective in the treatment of breast cancer, particularly HR-positive, HER2-negative advanced or metastatic breast cancer. The amounts, which together are effective, will relieve to some extent one or more of the symptoms of the disorder being treated. In reference to the treatment of cancer, a effective amount refers to that amount which has the effect of (1) reducing the size of the tumor, (2) inhibiting (that is, slowing to some extent, preferably stopping) tumor metastasis emergence, (3) inhibiting to some extent (that is, slowing to some extent, preferably stopping) tumor growth or tumor invasiveness, and/or (4) relieving to some extent (or, preferably, eliminating) one or more signs or symptoms associated with the cancer. Therapeutic or pharmacological effectiveness of the doses and administration regimens may also be characterized as the ability to induce, enhance, maintain or prolong disease control and/or overall survival in patients with these specific tumors, which may be measured as prolongation of the time before disease progression”.

In an embodiment, the invention is related to a method for treating breast cancer, particularly HR-positive, HER2-negative advanced or metastatic breast cancer comprising administering to a patient in need thereof an amount of a TGFβ inhibitor in combination with an amount of a CDK inhibitor, that is effective in treating breast cancer, particularly HR-positive, HER2-negative advanced or metastatic breast cancer. In a further embodiment, the invention is related to a method for treating breast cancer, particularly HR-positive, HER2-negative advanced or metastatic breast cancer comprising administering to a patient in need thereof an amount of a TGFβ inhibitor and an amount of a CDK inhibitor, wherein the amounts together are effective in treating breast cancer, particularly HR-positive, HER2-negative advanced or metastatic breast cancer. In another embodiment, the invention is related to combination of a TGFβ inhibitor and a CDK inhibitor, for use in the treatment of breast cancer, particularly HR-positive, HER2-negative advanced or metastatic breast cancer. In another embodiment, the invention is related to a method for treating breast cancer, particularly HR-positive,

HER2-negative advanced or metastatic breast cancer comprising administering to a patient in need thereof an amount of a TGFβ inhibitor and an amount of a CDK inhibitor, wherein the amounts together achieve synergistic effects in the treatment of breast cancer, particularly HR-positive, HER2-negative advanced or metastatic breast cancer. In another embodiment, the invention is related to a combination of a TGFβ inhibitor and a CDK inhibitor for the treatment of breast cancer, particularly HR-positive, HER2-negative advanced or metastatic breast cancer, wherein the combination is synergistic. In an embodiment, the method or use of the invention is related to a synergistic combination of targeted therapeutic agents, specifically a TGFβ inhibitor and a CDK inhibitor.

In an embodiment, the invention is related to a method for treating breast cancer, particularly HR-positive, HER2-negative advanced or metastatic breast cancer comprising administering to a patient in need thereof an amount of a TGFβ inhibitor in combination with an amount of a CDK inhibitor, that is effective in treating breast cancer, particularly HR-positive, HER2-negative advanced or metastatic breast cancer. In a further embodiment, the invention is related to a method for treating breast cancer, particularly HR-positive, HER2-negative advanced or metastatic breast cancer comprising administering to a patient in need thereof an amount of a TGFβ inhibitor and an amount of a CDK inhibitor, wherein the amounts together are effective in treating breast cancer, particularly HR-positive, HER2-negative advanced or metastatic breast cancer. In another embodiment, the invention is related to a combination of a TGFβ inhibitor and a CDK inhibitor for use in the treatment of breast cancer, particularly HR-positive, HER2-negative advanced or metastatic breast cancer. In another embodiment, the invention is related to a method for treating breast cancer, particularly HR-positive, HER2-negative advanced or metastatic breast cancer comprising administering to a patient in need thereof an amount of a TGFβ inhibitor and an amount of a CDK inhibitor, wherein the amounts together achieve synergistic effects in the treatment of breast cancer, particularly HR-positive, HER2-negative advanced or metastatic breast cancer. In another embodiment, the invention is related to a combination of a TGFβ inhibitor and a CDK inhibitor for the treatment of breast cancer, particularly HR-positive, HER2-negative advanced or metastatic breast cancer, wherein the combination is synergistic. In an embodiment, the method or use of the invention is related to a synergistic combination of targeted therapeutic agents, specifically a TGFβ inhibitor and a CDK inhibitor.

In an embodiment, the invention is related to a method for treating breast cancer, particularly HR-positive, HER2-negative advanced or metastatic breast cancer comprising administering to a patient in need thereof an amount of a TGFβ inhibitor in combination with an amount of a CDK 4/6 inhibitor, that is effective in treating breast cancer, particularly HR-positive, HER2-negative advanced or metastatic breast cancer. In a further embodiment, the invention is related to a method for treating breast cancer, particularly HR-positive, HER2-negative advanced or metastatic breast cancer comprising administering to a patient in need thereof an amount of a TGFβ inhibitor and an amount of a CDK 4/6 inhibitor, wherein the amounts together are effective in treating breast cancer, particularly HR-positive, HER2-negative advanced or metastatic breast cancer. In another embodiment, the invention is related to a combination of a TGFβ inhibitor and a CDK 4/6 inhibitor in the treatment of breast cancer, particularly HR-positive, HER2-negative advanced or metastatic breast cancer. In another embodiment, the invention is related to a method for treating breast cancer, particularly HR-positive, HER2-negative advanced or metastatic breast cancer comprising administering to a patient in need thereof an amount of a TGFβ inhibitor and an amount of a CDK 4/6 inhibitor, wherein the amounts together achieve synergistic effects in the treatment of breast cancer, particularly HR-positive, HER2-negative advanced or metastatic breast cancer. In another embodiment, the invention is related to a combination of a TGFβ inhibitor and a CDK 4/6 inhibitor for the treatment of breast cancer, particularly HR-positive, HER2-negative advanced or metastatic breast cancer, wherein the combination is synergistic. In an embodiment, the method or use of the invention is related to a synergistic combination of targeted therapeutic agents, specifically a TGFβ inhibitor and a CDK 4/6 inhibitor.

In an embodiment, the invention is related to a method for treating breast cancer, particularly HR-positive, HER2-negative advanced or metastatic breast cancer comprising administering to a patient in need thereof an amount of a TGFβ inhibitor in combination with an amount of a CDK 4/6 inhibitor, that is effective in treating breast cancer, particularly HR-positive, HER2-negative advanced or metastatic breast cancer. In a further embodiment, the invention is related to a method for treating breast cancer, particularly HR-positive, HER2-negative advanced or metastatic breast cancer comprising administering to a patient in need thereof an amount of a TGFβ inhibitor and an amount of a CDK 4/6 inhibitor, wherein the amounts together are effective in treating breast cancer, particularly HR-positive, HER2-negative advanced or metastatic breast cancer. In another embodiment, the invention is related to a combination of a TGFβ inhibitor and a CDK 4/6 inhibitor in the treatment of breast cancer, particularly HR-positive, HER2-negative advanced or metastatic breast cancer. In another embodiment, the invention is related to a method for treating breast cancer, particularly HR-positive, HER2-negative advanced or metastatic breast cancer comprising administering to a patient in need thereof an amount of a TGFβ inhibitor and an amount of a CDK 4/6 inhibitor, wherein the amounts together achieve synergistic effects in the treatment of breast cancer, particularly HR-positive, HER2-negative advanced or metastatic breast cancer. In another embodiment, the invention is related to a combination of a TGFβ inhibitor and a CDK 4/6 inhibitor for the treatment of breast cancer, particularly HR-positive, HER2-negative advanced or metastatic breast cancer, wherein the combination is synergistic. In an embodiment, the method or use of the invention is related to a synergistic combination of targeted therapeutic agents, specifically a TGFβ inhibitor and a CDK 4/6 inhibitor.

In an embodiment, the invention is related to a method for treating breast cancer, particularly HR-positive, HER2-negative advanced or metastatic breast cancer comprising administering to a patient in need thereof an amount of 4-(2-(5-chloro-2-fluorophenyl)-5-isopropylpyridin-4-ylamino)-N-(1,3-dihydroxypropan-2-Anicotinamide (PF-06952229), or a pharmaceutically acceptable salt thereof, in combination with an amount of palbociclib, or a pharmaceutically acceptable salt thereof, that is effective in treating breast cancer, particularly HR-positive, HER2-negative advanced or metastatic breast cancer. In a further embodiment, the invention is related to a method for treating breast cancer, particularly HR-positive, HER2-negative advanced or metastatic breast cancer comprising administering to a patient in need thereof an amount of 4-(2-(5-chloro-2-fluorophenyl)-5-isopropylpyridin-4-ylamino)-N-(1,3-dihydroxypropan-2-yl)nicotinamide (PF-06952229), or a pharmaceutically acceptable salt thereof, and an amount of palbociclib, or a pharmaceutically acceptable salt thereof, wherein the amounts together are effective in treating breast cancer, particularly HR-positive, HER2-negative advanced or metastatic breast cancer. In another embodiment, the invention is related to a combination of 4-(2-(5-chloro-2-fluorophenyl)-5-isopropylpyridi n-4-ylami no)-N-(1,3-dihydroxypropan-2-yl)nicotinamide (PF-06952229), or a pharmaceutically acceptable salt thereof, and palbociclib, or a pharmaceutically acceptable salt thereof, wherein the amounts together are effective in treating breast cancer, particularly HR-positive, HER2-negative advanced or metastatic breast cancer. In another embodiment, the invention is related to a method for treating breast cancer, particularly HR-positive, HER2-negative advanced or metastatic breast cancer comprising administering to a patient in need thereof an amount of 4-(2-(5-chloro-2-fluorophenyl)-5-isopropylpyridin-4-ylamino)-N-(1,3-dihydroxypropan-2-yl)nicotinamide (PF-06952229), or a pharmaceutically acceptable salt thereof, and an amount of palbociclib, or a pharmaceutically acceptable salt thereof, wherein the amounts together achieve synergistic effects in the treatment of breast cancer, particularly HR-positive, HER2-negative advanced or metastatic breast cancer. In another embodiment, the invention is related to a combination of 4-(2-(5-chloro-2-fluorophenyl)-5-isopropylpyridin-4-ylamino)-N-(1,3-dihydroxypropan-2-yl)nicotinamide (PF-06952229), or a pharmaceutically acceptable salt thereof, and palbociclib, or a pharmaceutically acceptable salt thereof for the treatment of breast cancer, particularly HR-positive, HER2-negative advanced or metastatic breast cancer, wherein the combination is synergistic. In an embodiment, the method or use of the invention is related to a synergistic combination of targeted therapeutic agents, specifically 4-(2-(5-chloro-2-fluorophenyl)-5-isopropylpyridin-4-ylamino)- N-(1, 3-di hydroxypropan-2-yl)nicotinamide (PF-06952229), or a pharmaceutically acceptable salt thereof, and palbociclib, or a pharmaceutically acceptable salt thereof.

A “standard clinical dosing regimen,” as used herein, refers to a regimen for administering a substance, agent, compound, or composition, which is typically used in a clinical setting. A “standard clinical dosing regimen,” includes a “standard clinical dose” or a “standard dosing schedule”.

A “non-standard clinical dosing regimen,” as used herein, refers to a regimen for administering a substance, agent, compound, or composition, which is different than the amount, dose or schedule typically used in a clinical setting. A “non-standard clinical dosing regimen,” includes a “non-standard clinical dose” or a “non-standard dosing schedule”.

In an embodiment, the invention is related to a method for treating breast cancer, particularly HR-positive, HER2-negative advanced or metastatic breast cancer comprising administering to a patient in need thereof an amount of a TGFβ inhibitor in combination with an amount of a CDK inhibitor, that is effective in treating breast cancer, particularly HR-positive, HER2-negative advanced or metastatic breast cancer, wherein the CDK inhibitor is administered according to a non-standard clinical dosing regimen. In a further embodiment, the invention is related to a method for treating breast cancer, particularly HR-positive, HER2-negative advanced or metastatic breast cancer comprising administering to a patient in need thereof an amount of a TGFβ inhibitor and an amount of a CDK inhibitor, wherein the CDK inhibitor is administered according to a non-standard clinical dosing regimen, and further wherein the amounts together are effective in treating breast cancer, particularly HR-positive, HER2-negative advanced or metastatic breast cancer. In another embodiment, the invention is related to a combination of a TGFβ inhibitor and an amount of a CDK inhibitor for use in the treatment of breast cancer, particularly HR-positive, HER2-negative advanced or metastatic breast cancer, wherein the CDK inhibitor is administered according to a non-standard clinical dosing regimen. In another embodiment, the invention is related to a method for treating breast cancer, particularly HR-positive, HER2-negative advanced or metastatic breast cancer comprising administering to a patient in need thereof an amount of a TGFβ inhibitor and an amount of a CDK inhibitor, wherein the CDK inhibitor is administered according to a non-standard clinical dosing regimen, and further wherein the amounts together achieve synergistic effects in treating breast cancer, particularly HR-positive, HER2-negative advanced or metastatic breast cancer. In another embodiment, the invention is related to a combination of a TGFβ inhibitor and a CDK inhibitor for the treatment of breast cancer, particularly HR-positive, HER2-negative advanced or metastatic breast cancer, wherein the CDK inhibitor is administered according to a non-standard clinical dosing regimen, and further wherein the combination is synergistic.

In an embodiment, the invention is related to a method for treating breast cancer, particularly HR-positive, HER2-negative advanced or metastatic breast cancer comprising administering to a patient in need thereof an amount of a TGFβ inhibitor in combination with an amount of a CDK inhibitor, that is effective in treating breast cancer, particularly HR-positive, HER2-negative advanced or metastatic breast cancer, wherein the CDK inhibitor is administered according to a non-standard clinical dosing regimen. In a further embodiment, the invention is related to a method for treating breast cancer, particularly HR-positive, HER2-negative advanced or metastatic breast cancer comprising administering to a patient in need thereof an amount of a TGFβ inhibitor and an amount of a CDK inhibitor, wherein the CDK inhibitor is administered according to a non-standard clinical dosing regimen, and further wherein the amounts together are effective in treating breast cancer, particularly HR-positive, HER2-negative advanced or metastatic breast cancer. In another embodiment, the invention is related to the use of a combination of a TGFβ inhibitor and a CDK inhibitor in the treatment of breast cancer, particularly HR-positive, HER2-negative advanced or metastatic breast cancer, wherein the CDK inhibitor is administered according to a non-standard clinical dosing regimen. In another embodiment, the invention is related to a method for treating breast cancer, particularly HR-positive, HER2-negative advanced or metastatic breast cancer comprising administering to a patient in need thereof an amount of a TGFβ inhibitor and an amount of a CDK inhibitor, wherein the CDK inhibitor is administered according to a non-standard clinical dosing regimen, and further wherein the amounts together achieve synergistic effects in treating breast cancer, particularly HR-positive, HER2-negative advanced or metastatic breast cancer. In another embodiment, the invention is related to the use of an amount a combination of a TG93 inhibitor and a CDK inhibitor for the treatment of breast cancer, particularly HR-positive, HER2-negative advanced or metastatic breast cancer, wherein the CDK inhibitor is administered according to a non-standard clinical dosing regimen.

In an embodiment, the invention is related to a method for treating breast cancer, particularly HR-positive, HER2-negative advanced or metastatic breast cancer comprising administering to a patient in need thereof an amount of a TGFβ inhibitor in combination with an amount of a CDK 4/6 inhibitor, that is effective in treating breast cancer, particularly HR-positive, HER2-negative advanced or metastatic breast cancer, wherein the CDK 4/6 inhibitor is administered according to a non-standard clinical dosing regimen. In a further embodiment, the invention is related to a method for treating breast cancer, particularly HR-positive, HER2-negative advanced or metastatic breast cancer comprising administering to a patient in need thereof an amount of a TGFβ inhibitor and an amount of a CDK 4/6 inhibitor, wherein the CDK 4/6 inhibitor is administered according to a non-standard clinical dosing regimen, and further wherein the amounts together are effective in treating n breast cancer, particularly HR-positive, HER2-negative advanced or metastatic breast cancer. In another embodiment, the invention is related to a combintaion of a TGFβ inhibitor and a CDK 4/6 inhibitor in the treatment of breast cancer, particularly HR-positive, HER2-negative advanced or metastatic breast cancer, wherein the CDK 4/6 inhibitor is administered according to a non-standard clinical dosing regimen. In another embodiment, the invention is related to a method for treating breast cancer, particularly HR-positive, HER2-negative advanced or metastatic breast cancer, comprising administering to a patient in need thereof an amount of a TGFβ inhibitor and an amount of a CDK 4/6 inhibitor, wherein the CDK 4/6 inhibitor is administered according to a non-standard clinical dosing regimen, and further wherein the amounts together achieve synergistic effects in treating breast cancer, particularly HR-positive, HER2-negative advanced or metastatic breast cancer. In another embodiment, the invention is related to a combination of a TGFβ inhibitor and a CDK 4/6 inhibitor for the treatment of breast cancer, particularly HR-positive, HER2-negative advanced or metastatic breast cancer, wherein the CDK 4/6 inhibitor is administered according to a non-standard clinical dosing regimen, and further wherein the combination is synergistic.

In an embodiment, the invention is related to a method for treating breast cancer, particularly HR-positive, HER2-negative advanced or metastatic breast cancer, comprising administering to a patient in need thereof an amount of a TGFβ inhibitor in combination with an amount of a CDK 4/6 inhibitor, that is effective in treating breast cancer, particularly HR-positive, HER2-negative advanced or metastatic breast cancer, wherein the CDK 4/6 inhibitor is administered according to a non-standard clinical dosing regimen. In a further embodiment, the invention is related to a method for treating breast cancer, particularly HR-positive, HER2-negative advanced or metastatic breast cancer comprising administering to a patient in need thereof an amount of a TGFβ inhibitor and an amount of a CDK 4/6 inhibitor, wherein the CDK 4/6 inhibitor is administered according to a non-standard clinical dosing regimen, and further wherein the amounts together are effective in treating breast cancer, particularly HR-positive, HER2-negative advanced or metastatic breast cancer. In another embodiment, the invention is related to a combination of a TGF8 inhibitor and an amount of a CDK 4/6 inhibitor for use in the treatment of breast cancer, particularly HR-positive, HER2-negative advanced or metastatic breast cancer, wherein the CDK 4/6 inhibitor is administered according to a non-standard clinical dosing regimen. In another embodiment, the invention is related to a method for treating breast cancer, particularly HR-positive, HER2-negative advanced or metastatic breast cancer comprising administering to a patient in need thereof an amount of a TGF8 inhibitor and an amount of a CDK 4/6 inhibitor, wherein the CDK 4/6 inhibitor is administered according to a non-standard clinical dosing regimen, and further wherein the amounts together achieve synergistic effects in treating breast cancer, particularly HR-positive, HER2-negative advanced or metastatic breast cancer. In another embodiment, the invention is related to a combination of a TGF8 inhibitor and a CDK 4/6 inhibitor for the treatment of breast cancer, particularly HR-positive, HER2-negative advanced or metastatic breast cancer, wherein the CDK 4/6 inhibitor is administered according to a non-standard clinical dosing regimen, and further wherein the combination is synergistic.

In an embodiment, the invention is related to a method for treating breast cancer, particularly HR-positive, HER2-negative advanced or metastatic breast cancer comprising administering to a patient in need thereof an amount of 4-(2-(5-chloro-2-fluorophenyl)-5-isopropylpyridin-4-ylamino)-N-(1,3-dihydroxypropan-2-yl)nicotinamide (PF-06952229), or a pharmaceutically acceptable salt thereof, in combination with an amount of palbociclib, or a pharmaceutically acceptable salt thereof, that is effective in treating breast cancer, particularly HR-positive, HER2-negative advanced or metastatic breast cancer, wherein the palbociclib, or a pharmaceutically acceptable salt thereof is administered according to a non-standard clinical dosing regimen. In a further embodiment, the invention is related to a method for treating breast cancer, particularly HR-positive, HER2-negative advanced or metastatic breast cancer comprising administering to a patient in need thereof an amount of 4-(2-(5-chloro-2-fluorophenyl)-5-isopropylpyridin-4-ylamino)-N-(1,3-dihydroxypropan-2-yl)nicotinamide (PF-06952229), or a pharmaceutically acceptable salt thereof, and an amount of a palbociclib, or a pharmaceutically acceptable salt thereof, wherein the palbociclib, or a pharmaceutically acceptable salt thereof is administered according to a non-standard clinical dosing regimen, and further wherein the amounts together are effective in treating breast cancer, particularly HR-positive, HER2-negative advanced or metastatic breast cancer. In another embodiment, the invention is related to a combination of 4-(2-(5-chloro-2-fluorophenyl)-5-isopropylpyridin-4-ylamino)-N-(1,3-dihydroxypropan-2-yl)nicotinamide (PF-06952229), or a pharmaceutically acceptable salt thereof, and palbociclib, or a pharmaceutically acceptable salt thereof, for use in the treatment of breast cancer, particularly HR-positive, HER2-negative advanced or metastatic breast cancer, wherein the palbociclib, or a pharmaceutically acceptable salt thereof is administered according to a non-standard clinical dosing regimen. In another embodiment, the invention is related to a method for treating breast cancer, particularly HR-positive, HER2-negative advanced or metastatic breast cancer comprising administering to a patient in need thereof an amount of 4-(2-(5-chloro-2-fluorophenyl)-5-isopropylpyridin-4-ylamino)-N-(1,3-dihydroxypropan-2-yl)nicotinamide (PF-06952229), or a pharmaceutically acceptable salt thereof, and an amount of palbociclib, or a pharmaceutically acceptable salt thereof, wherein the palbociclib, or a pharmaceutically acceptable salt thereof is administered according to a non-standard clinical dosing regimen, and further wherein the amounts together achieve synergistic effects in treating breast cancer, particularly HR-positive, HER2-negative advanced or metastatic breast cancer. In another embodiment, the invention is related to a combination of 4-(2-(5-chloro-2-fluorophenyl)-5-isopropylpyridin-4-ylamino)-N-(1,3-dihydroxypropan-2-yl)nicotinamide (PF-06952229), or a pharmaceutically acceptable salt thereof, and palbociclib, or a pharmaceutically acceptable salt thereof, for the treatment of breast cancer, particularly HR-positive, HER2-negative advanced or metastatic breast cancer, wherein the palbociclib, or a pharmaceutically acceptable salt thereof is administered according to a non-standard clinical dosing regimen, and further wherein the combination is synergistic.

A “low-dose amount”, as used herein, refers to an amount or dose of a substance, agent, compound, or composition, that is lower than the amount or dose typically used in a clinical setting.

In an embodiment, the invention is related to a method for treating breast cancer, particularly HR-positive, HER2-negative advanced or metastatic breast cancer comprising administering to a patient in need thereof an amount of a TGFβ inhibitor in combination with a low-dose amount of a CDK inhibitor, that is effective in treating breast cancer, particularly HR-positive, HER2-negative advanced or metastatic breast cancer. In a further embodiment, the invention is related to a method for treating breast cancer, particularly HR-positive, HER2-negative advanced or metastatic breast cancer comprising administering to a patient in need thereof an amount of a TGFβ inhibitor and a low-dose amount of a CDK inhibitor, wherein the amounts together are effective in treating breast cancer, particularly HR-positive, HER2-negative advanced or metastatic breast cancer. In another embodiment, the invention is related to a combination of a TGFβ inhibitor and a low-dose amount of a CDK inhibitor for use in the treatment of breast cancer, particularly HR-positive, HER2-negative advanced or metastatic breast cancer. In another embodiment, the invention is related to a method for treating breast cancer, particularly HR-positive, HER2-negative advanced or metastatic breast cancer comprising administering to a patient in need thereof an amount of a TGFβ inhibitor and a low-dose amount of a CDK inhibitor, wherein the amounts together achieve synergistic effects in the treatment of breast cancer, particularly HR-positive, HER2-negative advanced or metastatic breast cancer. In another embodiment, the invention is related to a combination of a TG93 inhibitor and a low-dose amount of a CDK inhibitor for the treatment of breast cancer, particularly HR-positive, HER2-negative advanced or metastatic breast cancer, wherein the combination is synergistic.

In an embodiment, the invention is related to a method for treating breast cancer, particularly HR-positive, HER2-negative advanced or metastatic breast cancer comprising administering to a patient in need thereof an amount of a TGFβ inhibitor in combination with a low-dose amount of a CDK inhibitor, that is effective in treating breast cancer, particularly HR-positive, HER2-negative advanced or metastatic breast cancer. In a further embodiment, the invention is related to a method for treating breast cancer, particularly HR-positive, HER2-negative advanced or metastatic breast cancer comprising administering to a patient in need thereof an amount of a TGFβ inhibitor and a low-dose amount of a CDK inhibitor, wherein the amounts together are effective in treating breast cancer, particularly HR-positive, HER2-negative advanced or metastatic breast cancer. In another embodiment, the invention is related to a combination of a TGFβ inhibitor and a low-dose amount of a CDK inhibitor for use in the treatment of breast cancer, particularly HR-positive, HER2-negative advanced or metastatic breast cancer. In another embodiment, the invention is related to a method for treating breast cancer, particularly HR-positive, HER2-negative advanced or metastatic breast cancer comprising administering to a patient in need thereof an amount of a TGFβ inhibitor and a low-dose amount of a CDK inhibitor, wherein the amounts together achieve synergistic effects in the treatment of breast cancer, particularly HR-positive, HER2-negative advanced or metastatic breast cancer. In another embodiment, the invention is related to a combination of a TG93 inhibitor and a low-dose amount of a CDK inhibitor for the treatment of breast cancer, particularly HR-positive, HER2-negative advanced or metastatic breast cancer, wherein the combination is synergistic.

In an embodiment, the invention is related to a method for treating breast cancer, particularly HR-positive, HER2-negative advanced or metastatic breast cancer comprising administering to a patient in need thereof an amount of a TGFβ inhibitor in combination with a low-dose amount of a CDK 4/6 inhibitor, that is effective in treating breast cancer, particularly HR-positive, HER2-negative advanced or metastatic breast cancer. In a further embodiment, the invention is related to a method for treating breast cancer, particularly HR-positive, HER2-negative advanced or metastatic breast cancer comprising administering to a patient in need thereof an amount of a TGFβ inhibitor and a low-dose amount of a CDK 4/6 inhibitor, wherein the amounts together are effective in treating breast cancer, particularly HR-positive, HER2-negative advanced or metastatic breast cancer. In another embodiment, the invention is related to a combination of a TGFβ inhibitor and a low-dose amount of a CDK 4/6 inhibitor for use in the treatment of breast cancer, particularly HR-positive, HER2-negative advanced or metastatic breast cancer. In another embodiment, the invention is related to a method for treating breast cancer, particularly HR-positive, HER2-negative advanced or metastatic breast cancer comprising administering to a patient in need thereof an amount of a TGFβ inhibitor and a low-dose amount of a CDK 4/6 inhibitor, wherein the amounts together achieve synergistic effects in the treatment of breast cancer, particularly HR-positive, HER2-negative advanced or metastatic breast cancer. In another embodiment, the invention is related to a combination of a TGFβ inhibitor and a low-dose amount of a CDK 4/6 inhibitor for the treatment of breast cancer, particularly HR-positive, HER2-negative advanced or metastatic breast cancer, wherein the combination is synergistic.

In an embodiment, the invention is related to a method for treating breast cancer, particularly HR-positive, HER2-negative advanced or metastatic breast cancer comprising administering to a patient in need thereof an amount of a TGFβ inhibitor in combination with a low-dose amount of a CDK 4/6 inhibitor, that is effective in treating breast cancer, particularly HR-positive, HER2-negative advanced or metastatic breast cancer. In a further embodiment, the invention is related to a method for treating breast cancer, particularly HR-positive, HER2-negative advanced or metastatic breast cancer comprising administering to a patient in need thereof an amount of a TGFβ inhibitor and a low-dose amount of a CDK 4/6 inhibitor, wherein the amounts together are effective in treating breast cancer, particularly HR-positive, HER2-negative advanced or metastatic breast cancer. In another embodiment, the invention is related to a combinatoin of a TGFβ inhibitor and a low-dose amount of a CDK 4/6 inhibitor for use in the treatment of breast cancer, particularly HR-positive, HER2-negative advanced or metastatic breast cancer. In another embodiment, the invention is related to a method for treating breast cancer, particularly HR-positive, HER2-negative advanced or metastatic breast cancer comprising administering to a patient in need thereof an amount of a TGFβ inhibitor and a low-dose amount of a CDK 4/6 inhibitor, wherein the amounts together achieve synergistic effects in the treatment of breast cancer, particularly HR-positive, HER2-negative advanced or metastatic breast cancer. In another embodiment, the invention is related to a combination of a TGFβ inhibitor and a low-dose amount of a CDK 4/6 inhibitor for the treatment of breast cancer, particularly HR-positive, HER2-negative advanced or metastatic breast cancer, wherein the combination is synergistic.

In an embodiment, the invention is related to a method for treating breast cancer, particularly HR-positive, HER2-negative advanced or metastatic breast cancer comprising administering to a patient in need thereof an amount of 4-(2-(5-chloro-2-fluorophenyl)-5-isopropylpyridin-4-ylamino)-N-(1,3-dihydroxypropan-2-yl)nicotinamide (PF-06952229), or a pharmaceutically acceptable salt thereof, in combination with a low-dose amount of palbociclib, or a pharmaceutically acceptable salt thereof, that is effective in treating breast cancer, particularly HR-positive, HER2-negative advanced or metastatic breast cancer. In a further embodiment, the invention is related to a method for treating breast cancer, particularly HR-positive, HER2-negative advanced or metastatic breast cancer comprising administering to a patient in need thereof an amount of 4-(2-(5-chloro-2-fluorophenyl)-5-isopropylpyridin-4-ylamino)-N-(1,3-dihydroxypropan-2-yl)nicotinamide (PF-06952229), or a pharmaceutically acceptable salt thereof, and a low-dose amount of palbociclib, or a pharmaceutically acceptable salt thereof, wherein the amounts together are effective in treating breast cancer, particularly HR-positive, HER2-negative advanced or metastatic breast cancer. In another embodiment, the invention is related to a combination of 4-(2-(5-chloro-2-fluorophenyl)-5-isopropylpyridin-4-ylamino)-N-(1,3-dihydroxypropan-2-Anicotinamide (PF-06952229), or a pharmaceutically acceptable salt thereof, and a low-dose amount of a palbociclib, or a pharmaceutically acceptable salt thereof, for use in the treatment of breast cancer, particularly HR-positive, HER2-negative advanced or metastatic breast cancer. In another embodiment, the invention is related to a method for treating breast cancer, particularly HR-positive, HER2-negative advanced or metastatic breast cancer comprising administering to a patient in need thereof an amount of 4-(2-(5-chloro-2-fluorophenyl)-5-isopropylpyridin-4-ylamino)-N-(1,3-dihydroxypropan-2-yl)nicotinamide (PF-06952229), or a pharmaceutically acceptable salt thereof, and a low-dose amount of a palbociclib, or a pharmaceutically acceptable salt thereof, wherein the amounts together achieve synergistic effects in the treatment of breast cancer, particularly HR-positive, HER2-negative advanced or metastatic breast cancer. In another embodiment, the invention is related to a combination of an amount of 4-(2-(5-chloro-2-fluorophenyl)-5-isopropylpyridin-4-ylamino)-N-(1,3-dihydroxypropan-2-yl)nicotinamide (PF-06952229), or a pharmaceutically acceptable salt thereof, and a low-dose amount of palbociclib, or a pharmaceutically acceptable salt thereof, for the treatment of breast cancer, particularly HR-positive, HER2-negative advanced or metastatic breast cancer, wherein the combination is synergistic.

Those skilled in the art will be able to determine, according to known methods, the appropriate amount, dose or dosage of each compound, as used in the combination of the present invention, to administer to a patient, taking into account factors such as age, weight, general health, the compound administered, the route of administration, the nature and advancement of the breast cancer, particularly HR-positive, HER2-negative advanced or metastatic breast cancer, requiring treatment, and the presence of other medications.

In an embodiment, palbociclib, or a pharmaceutically acceptable salt thereof, is administered at a daily dosage of about 125 mg once daily, about 100 mg once daily, about 75 mg once daily, or about 50 mg daily. In an embodiment, which is the recommended starting dose or standard clinical dose, palbociclib, or a pharmaceutically acceptable salt thereof, is administered at a daily dosage of about 125 mg once a day. In an embodiment, palbociclib, or a pharmaceutically acceptable salt thereof, is administered at a non-standard clinical dose. In an embodiment, a non-standard clinical dose is a low-dose amount of palbociclib, or a pharmaceutically acceptable salt thereof. For example, palbociclib, or a pharmaceutically acceptable salt thereof, is administered at a dose of about 100 mg once daily, about 75 mg once daily, or about 50 mg once daily. In an embodiment, palbociclib, or a pharmaceutically acceptable salt thereof, is administered at a dose of about 100 mg once daily. In an embodiment, palbociclib, or a pharmaceutically acceptable salt thereof, is administered at a dose of about 75 mg once daily. In an embodiment, palbociclib, or a pharmaceutically acceptable salt thereof, is administered at a dose of about 50 mg once daily. Dosage amounts provided herein refer to the dose of the free base form of palbociclib, or are calculated as the free base equivalent of an administered palbociclib salt form. For example, a dosage or amount of palbociclib, such as 100 mg, 75 mg or 50 mg, refers to the free base equivalent. This dosage regimen may be adjusted to provide the optimal therapeutic response. For example, the dose may be proportionally reduced or increased as indicated by the exigencies of the therapeutic situation.

In an embodiment, PF-06873600, or a pharmaceutically acceptable salt thereof, is administered at a daily dosage of about 125 mg once daily, about 100 mg once daily, about 75 mg once daily, or about 50 mg daily. In an embodiment, PF-06873600, or a pharmaceutically acceptable salt thereof, is administered at a daily dosage of about 125 mg once a day. In an embodiment, PF-06873600, or a pharmaceutically acceptable salt thereof, is administered at a non-standard clinical dose. In an embodiment, a non-standard clinical dose is a low-dose amount of PF-06873600, or a pharmaceutically acceptable salt thereof. For example, PF-06873600, or a pharmaceutically acceptable salt thereof, is administered at a dose of about 100 mg once daily, about 75 mg once daily, or about 50 mg once daily. In an embodiment, PF-06873600, or a pharmaceutically acceptable salt thereof, is administered at a dose of about 100 mg once daily. In an embodiment, PF-06873600, or a pharmaceutically acceptable salt thereof, is administered at a dose of about 75 mg once daily. In an embodiment, PF-06873600, or a pharmaceutically acceptable salt thereof, is administered at a dose of about 50 mg once daily. Dosage amounts provided herein refer to the dose of the free base form of PF-06873600, or are calculated as the free base equivalent of an administered PF-06873600 salt form. For example, a dosage or amount of PF-06873600, such as 100 mg, 75 mg or 50 mg, refers to the free base equivalent. This dosage regimen may be adjusted to provide the optimal therapeutic response. For example, the dose may be proportionally reduced or increased as indicated by the exigencies of the therapeutic situation.

The practice of the method of this invention may be accomplished through various administration or dosing regimens. The compounds of the combination of the present invention can be administered intermittently, concurrently or sequentially. In an embodiment, the compounds of the combination of the present invention can be administered in a concurrent dosing regimen.

Repetition of the administration or dosing regimens may be conducted as necessary to achieve the desired reduction or diminution of cancer cells. A “continuous dosing schedule”, as used herein, is an administration or dosing regimen without dose interruptions, e.g., without days off treatment. Repetition of 21 or 28 day treatment cycles without dose interruptions between the treatment cycles is an example of a continuous dosing schedule. In an embodiment, the compounds of the combination of the present invention can be administered in a continuous dosing schedule. In an embodiment, the compounds of the combination of the present invention can be administered concurrently in a continuous dosing schedule.

In an embodiment, PF-06952229, or a pharmaceutically acceptable salt thereof, is administered once daily to comprise a complete cycle of 28 days. Repetition of the 28 day cycles is continued during treatment with the combination of the present invention.

In an embodiment, PF-06952229, or a pharmaceutically acceptable salt thereof, is administered once daily to comprise a complete cycle of 21 days. Repetition of the 21 day cycles is continued during treatment with the combination of the present invention.

The standard recommended dosing regimen, which includes the standard dosing schedule, for palbociclib, or a pharmaceutically acceptable salt thereof, is administration once daily for 21 consecutive days followed by 7 days off treatment to comprise a complete cycle of 28 days. Repetition of the 28 day cycles is continued during treatment with the combination of the present invention.

In an embodiment, palbociclib, or a pharmaceutically acceptable salt thereof, is administered under a non-standard dosing schedule. For example, palbociclib, or a pharmaceutically acceptable salt thereof, is administered once daily to comprise a complete cycle of 28 days. Repetition of the 28 day cycles is continued during treatment with the combination of the present invention.

In an embodiment, palbociclib, or a pharmaceutically acceptable salt thereof, is administered under a non-standard dosing schedule. For example, palbociclib, or a pharmaceutically acceptable salt thereof, is administered once daily to comprise a complete cycle of 21 days. Repetition of the 21 day cycles is continued during treatment with the combination of the present invention.

In an embodiment, palbociclib, or a pharmaceutically acceptable salt thereof, is administered under a non-standard dosing schedule. For example, palbociclib, or a pharmaceutically acceptable salt thereof, is administered once daily for 14 consecutive days followed by 7 days off treatment to comprise a complete cycle of 21 days. Repetition of the 21 day cycles is continued during treatment with the combination of the present invention.

The standard clinical dosing regimen, for palbociclib, or a pharmaceutically acceptable salt thereof, is administration of 125 mg once daily for 21 consecutive days followed by 7 days off treatment to comprise a complete cycle of 28 days. Repetition of the 28 day cycles is continued during treatment with the combination of the present invention.

In an embodiment, palbociclib, or a pharmaceutically acceptable salt thereof, is administered under a non-standard clinical dosing regimen. For example, palbociclib, or a pharmaceutically acceptable salt thereof, is administered at about 50 mg, about 75 mg or about 100 mg once daily to comprise a complete cycle of 28 days. Repetition of the 28 day cycles is continued during treatment with the combination of the present invention. In an embodiment, palbociclib, or a pharmaceutically acceptable salt thereof, is administered at about 50 mg. In an embodiment, palbociclib, or a pharmaceutically acceptable salt thereof, is administered at about 75 mg. In an embodiment, palbociclib, or a pharmaceutically acceptable salt thereof, is administered at about 100 mg.

In an embodiment, palbociclib, or a pharmaceutically acceptable salt thereof, is administered under a non-standard clinical dosing regimen. For example, palbociclib, or a pharmaceutically acceptable salt thereof, is administered at about 50 mg, about 75 mg or about 100 mg once daily to comprise a complete cycle of 21 days. Repetition of the 21 day cycles is continued during treatment with the combination of the present invention. In an embodiment, palbociclib, or a pharmaceutically acceptable salt thereof, is administered at about 50 mg. In an embodiment, palbociclib, or a pharmaceutically acceptable salt thereof, is administered at about 75 mg. In an embodiment, palbociclib, or a pharmaceutically acceptable salt thereof, is administered at about 100 mg.

In an embodiment, palbociclib, or a pharmaceutically acceptable salt thereof, is administered under a non-standard clinical dosing regimen. For example, palbociclib, or a pharmaceutically acceptable salt thereof, is administered at about 75 mg once daily for 14 consecutive days followed by 7 days off treatment to comprise a complete cycle of 21 days. Repetition of the 21 day cycles is continued during treatment with the combination of the present invention.

In one embodiment of the invention, PF-06952229 is administered at 20mg twice daily (BID), optionally employing a 7 days on/7 days off regimen in a 28 day cycle.

In one embodiment of the invention, PF-06952229 is administered at 40mg twice daily (BID), optionally employing a 7 days on/7 days off regimen in a 28 day cycle.

In one embodiment of the invention, PF-06952229 is administered at 80mg twice daily (BID), optionally employing a 7 days on/7 days off regimen in a 28 day cycle.

In one embodiment of the invention, PF-06952229 is administered at 150mg twice daily (BID), optionally employing a 7 days on/7 days off regimen in a 28 day cycle.

In one embodiment of the invention, PF-06952229 is administered at 250mg twice daily (BID), optionally employing a 7 days on/7 days off regimen in a 28 day cycle.

In one embodiment of the invention, PF-06952229 is administered at 375mg twice daily (BID), optionally employing a 7 days on/7 days off regimen in a 28 day cycle.

In one embodiment of the invention, PF-06952229 is administered at 500mg twice daily (BID), optionally employing a 7 days on/7 days off regimen in a 28 day cycle.

In one embodiment of the invention, PF-06952229 is administered at 625mg twice daily (BID), optionally employing a 7 days on/7 days off regimen in a 28 day cycle.

In further embodiments of the invention PF-06952229 is administered in combination with palbociclib and letrozole, where the palbociclib is administered at 125mg orally, once daily for 21 days followed by 7 days off, and where the letrozole is administered at 2.5mg orally, daily.

Administration of the compounds of the combination of the present invention can be effected by any method that enables delivery of the compounds to the site of action. These methods include oral routes, intraduodenal routes, parenteral injection (including intravenous, subcutaneous, intramuscular, intravascular or infusion), topical, and rectal administration.

The compounds of the method or combination of the present invention may be formulated prior to administration. The formulation will preferably be adapted to the particular mode of administration. These compounds may be formulated with pharmaceutically acceptable carriers as known in the art and administered in a wide variety of dosage forms as known in the art. In making the pharmaceutical compositions of the present invention, the active ingredient will usually be mixed with a pharmaceutically acceptable carrier, or diluted by a carrier or enclosed within a carrier. Such carriers include, but are not limited to, solid diluents or fillers, excipients, sterile aqueous media and various non-toxic organic solvents. Dosage unit forms or pharmaceutical compositions include tablets, capsules, such as gelatin capsules, pills, powders, granules, aqueous and nonaqueous oral solutions and suspensions, lozenges, troches, hard candies, sprays, creams, salves, suppositories, jellies, gels, pastes, lotions, ointments, injectable solutions, elixirs, syrups, and parenteral solutions packaged in containers adapted for subdivision into individual doses.

Parenteral formulations include pharmaceutically acceptable aqueous or nonaqueous solutions, dispersion, suspensions, emulsions, and sterile powders for the preparation thereof. Examples of carriers include water, ethanol, polyols (propylene glycol, polyethylene glycol), vegetable oils, and injectable organic esters such as ethyl oleate. Fluidity can be maintained by the use of a coating such as lecithin, a surfactant, or maintaining appropriate particle size. Exemplary parenteral administration forms include solutions or suspensions of the compounds of the invention in sterile aqueous solutions, for example, aqueous propylene glycol or dextrose solutions. Such dosage forms can be suitably buffered, if desired.

Additionally, lubricating agents such as magnesium stearate, sodium lauryl sulfate and talc are often useful for tableting purposes. Solid compositions of a similar type may also be employed in soft and hard filled gelatin capsules. Preferred materials, therefor, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

Methods of preparing various pharmaceutical compositions with a specific amount of active compound are known, or will be apparent, to those skilled in this art. For examples, see Remington's Pharmaceutical Sciences, Mack Publishing Company, Easter, Pa., 15th Edition (1975).

The invention also relates to a kit comprising the therapeutic agents of the combination of the present invention and written instructions for administration of the therapeutic agents. In one embodiment, the written instructions elaborate and qualify the modes of administration of the therapeutic agents, for example, for simultaneous or sequential administration of the therapeutic agents of the present invention. In one embodiment, the written instructions elaborate and qualify the modes of administration of the therapeutic agents, for example, by specifying the days of administration for each of the therapeutic agents during a 28 day cycle.

### EXAMPLES

### Example 1

### The TGFβ Inhibitor PF-06952229 Synergizes with a CDK4/6 Inhibitor Palbociclib and with a CDK2/4/6 Inhibitor (PF-068736000) in the CT26 Syngeneic Mouse Tumor Model

### Overview

PF-06952229 was evaluated in the CT26 syngeneic mouse tumor model in combination with palbociclib to assess efficacy on primary tumor growth and survival. PF-06952229 in combination with the CDK4/6 inhibitor palbociclib led to a significant increase in survival relative to PF-06952229 monotherapy (p=0.009) and to palbociclib monotherapy (p=0.017).

### Materials and Methods

CT26 cells were obtained from American Type Culture Collection (ATCC) and cultured in Roswell Park Memorial Institute (RPM11640) supplemented with 10% fetal bovine serum (FBS). All cells were maintained in a humidified incubator at 37° C. with 5% carbon dioxide (CO₂). Female Balb/cJ mice were obtained from Jackson Laboratories at 8 weeks of age. To generate the syngeneic model, 0.25 million CT26 tumor cells were subcutaneously implanted into the right flank of female BALB/cJ mice. Tumor bearing mice were randomized into six treatment groups based on average tumor sizes of approximately 50 mm³ per group, on Day 10 post tumor cell implantation. Study groups included vehicle, 30 mg/kg PF-06952229, 10 mg/kg PD-0332991 (Palbociclib), PF-06873600 (CDK 2/4/6 inhibitor), combination of PF-06952229 +PD-0332991 and combination of PF-06952229+PF-06873600. PF-06952229 was administered orally twice daily (BID) with 7 days on and 7 days off schedule. PD-0332991 or PF-06873600 was administered orally BID continuously, until the end of the study. The treatment groups and dose regimen information are summarized in Table 1:

TABLE 1

| | | Animals/ | | |
|---|---|---|---|---|
| Group | Drug | group | Route | Regimen |
| 1 | vehicle | 10 | PO | BID 7 days on, 7 days off |
| 2 | PD-0332991 (Palbociclib) | 10 | PO | BID continuously |
| | 10 mg/kg | | | |
| 3 | PF-06952229 30 mg/kg | 10 | PO | BID 7 days on, 7 days off |
| 4 | PD-0332991 (Palbociclib) | 10 | PO + PO | BID continuously + |
| | 10 mg/kg + | | | BID 7 days on 7 days off |
| | PF06952229 | | | |
| | 30 mg/kg | | | |
| 5 | PF-06873600 50 mg/kg | 10 | PO | BID continuously |
| 6 | PF-06873600 50 mg/kg + | 10 | PO + PO | BID continuously + |
| | PF06952229 30 mg/kg | | | BID 7 days on 7 days off |

BID = twice daily;
PO = oral dosing;

Tumor volumes were measured three times a week. Tumor volume was calculated based on two dimensional caliper measurement with cubic millimeter volume calculated using the formula (length×width2)×0.5. Mice were sacrificed when the tumor volumes reached 2000 mm³, which was the survival endpoint for this study. Survival curves were plotted using GraphPad Prism 7 software. Statistical analyses were performed using the Log-rank (Mantel-Cox) test.

### Results:

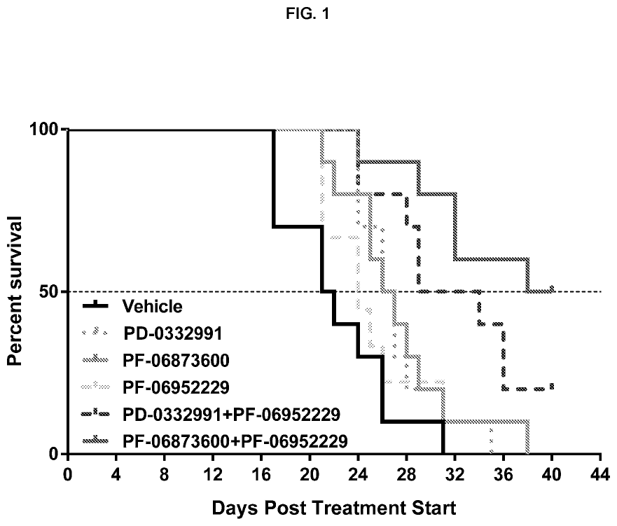

Survival results on Day 40 post-treatment initiation show that treatment with the TGFβ inhibitor PF-06952229 monotherapy did not significantly increase survival in the CT26 syngeneic tumor model; however, PF-06952229 treatment in combination with the CDK4/6 inhibitor palbociclib led to a significant increase in survival relative to PF-06952229 monotherapy (p=0.0088) and to palbociclib monotherapy (p=0.0173). A significant combinatorial effect was also observed when the TGFβ inhibitor PF-06952229 was combined with the CDK2/4/6 inhibitor PF-06873600, leading to significant increase in survival relative to PF-06952229 monotherapy (p<0.0001), and to PF-06873600 monotherapy (p=0.0013) See FIG. 1, and Table 2:

TABLE 2

| | | Median | P values | P values | P values | P values |
|---|---|---|---|---|---|---|
| | | Survival | (vs | (vs | (vs | (vs |
| Group | Agent | (Days) | vehicle) | PF-06952229) | Palbociclib) | PF-06873600) |
| 1 | Vehicle | 21.5 | N/A | 0.34 | 0.035 | 0.071 |
| 2 | PF-06952229 | 24 | 0.34 | N/A | 0.1294 | <0.0001 |
| 3 | Palbociclib | 26.5 | 0.035 | 0.1294 | N/A | N/A |
| 4 | PF-06952229 + | 31.5 | 0.0009 | 0.0088 | 0.0173 | N/A |
| | Palbociclib | | | | | |
| 5 | PF-06873600 | 26.5 | 0.071 | <0.0001 | N/A | N/A |
| 6 | PF-06952229 + | 39 | <0.0001 | 0.0003 | N/A | 0.0013 |
| | PF-06873600 | | | | | |

Statistical analyses were performed using Log-rank (Mantel-Cox) test. P values <0.05 are considered statistically significant; N/A = Not applicable

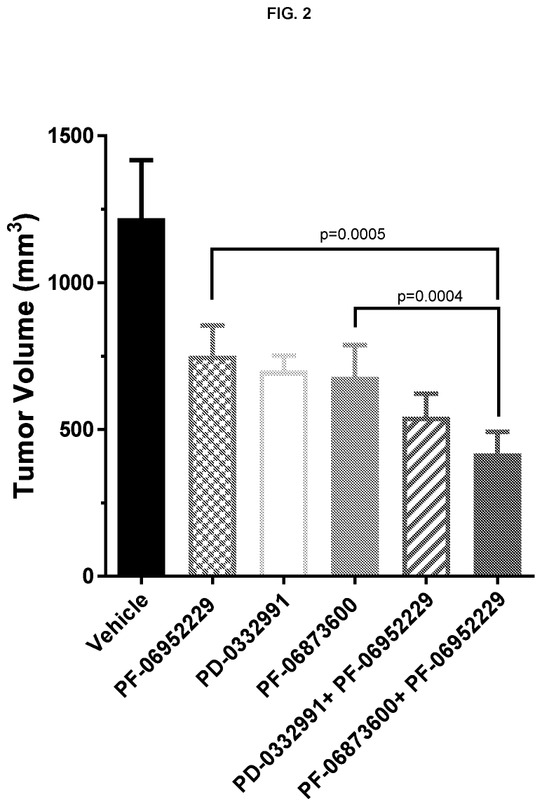

On Day 17 post-treatment initiation, tumor growth results show that treatment with the TGFβ inhibitor PF-06952229 monotherapy did not significantly inhibit tumor growth in the CT26 xenograft tumor model; however, PF-06952229 treatment in combination with the CDK 2/4/6 inhibitor PF-06873600 led to a significant combinatorial effect and thus an increase in tumor growth inhibition relative to PF-06952229 monotherapy (p=0.0005) and to PF-06873600 monotherapy (p=0.0004) (FIG. 2). Similarly, the combination of PF-06952229 with palbociclib (PD-0332991) also showed a trend to a combinatorial effect, with increase in tumor growth inhibition, when compared PF-06952229 or palbociclib monotherapy treatments alone (FIG. 2).

### Conclusions

TGFβ inhibitor PF 06952229 combination with the CDK4/6 inhibitor palbociclib or the CDK2/4/6 inhibitor led to greater tumor growth inhibition and significant improvement in survival relative to PF-06952229 monotherapy or CDK inhibitors monotherpaies, in the CT26 syngeneic tumor model.

### Example 2

### PF-06952229 Synergizes with Palbociclib and Pabociclib+Fulvestrant in the MCF7 Human ER+ Xenograft Mouse Tumor Model

### Overview

PF-06952229 was evaluated in the MCF-7 ER⁺ HER2⁻ breast cancer tumor mouse model mice in combination with the CDK 4/6 inhibitor palbociclib in absence or presence of the selective estrogen receptor degrader, fulvestrant. PF-06952229 combination with the CDK4/6 inhibitor palbociclib (PD-0332991) led to significant inhibition of tumor growth relative to either monotherapy alone. Similar results were observed when PF-06952229 was combined to palbociclib plus fulvestrant.

### Materials and Methods

MCF7 human ER⁺ breast cancer cells were obtained from American Type Culture

Collection (ATCC) and cultured in Roswell Park Memorial Institute (RPMI1640) supplemented with 10% fetal bovine serum (FBS). All cells were maintained in a humidified incubator at 37° C. with 5% carbon dioxide (CO₂). Female NSG mice were obtained from Jackson Laboratories at 7 weeks of age. To generate the xenograft model, 17β-ESTRADIOL pellets (0.36 mg, 90-day release) were subcutaneously implanted into the left flank of female NSG mice, 7 days before the tumor cell implantation. Then 5 million MCF7 cancer cells were subcutaneously implanted into the right axial region of female NSG mice. Tumor-bearing mice were randomized into treatment groups based on average tumor sizes of approximately 180 mm³, on Day 27 post-tumor cell implantation, and treatments were initiated. Treatment groups included vehicle, 10mg/kg PD-0332991, 30 mg/kg PF-06952229, PD-0332991+PF-05279929 (10 mg/kg), PF-06952229+PD-0332991, and the triple combination of PF-06952229+PD-0332991+PF-05279929. PF-06952229 was administered orally twice daily (BID) with 7 days on and 7 days off schedule. PD-0332991 was administered orally BID continuously until the end of the study. PF-05279929 was administered subcutaneously twice per week. The treatment groups and dose regimen information are summarized in Table 3:

TABLE 3

| | | Animals/ | | |
|---|---|---|---|---|
| Group | Drug | group | Route | Regimen |
| 1 | vehicle | 15 | PO | BID 7 on, 7 off |
| 2 | PD-0332991 10 mg/kg | 15 | PO | BID continuously |
| 3 | PF-06952229 30 mg/kg | 15 | PO | BID 7 days on, 7 days off |
| 4 | PD-0332991 10 mg/kg + | 15 | PO + SC | BID continuously + |
| | PF-05279929 10 mg/kg | | | twice per week |
| 5 | PF-06952229 30 mg/kg + | 15 | PO + PO | BID 7 days on, 7 days off + |
| | PD-0332991 10 mg/kg | | | BID continuously |
| 6 | PF-06952229 30 mg/kg + | 15 | PO + PO + SC | BID 7 days on, 7 days off + |
| | PD-0332991 10 mg/kg + | | | BID continuously + |
| | PF-05279929 10 mg/kg | | | twice per week |

BID = twice daily;
PO = oral dosing;
SC = subcutaneous dosing

Tumor volumes were measured two times a week. Tumor volume was calculated based on two-dimensional caliper measurement with cubic millimeter volume calculated using the formula (length×width²)×0.5. Body weights were measured two times a week. Tumor growth curves were plotted using GraphPad Prism 7 software. Statistical analysis of covariance (ANCOVA) model was applied to evaluate the treatment effect on tumor size at each time point post treatment, adjusting for the baseline tumor size of individual animals. Comparisons of treated groups to control group or to other treated groups are made using a t statistic under the ANCOVA model with fold change and the associated 95% confidence interval calculated.

pSMAD2 Bioassay: Tumor samples were collected and snap-frozen in 2.0 mL cryogenic tubes (Nalgene™) prior to analysis. Thawed tumor samples were homogenized in cell extraction buffer (Invitrogen, Carlsbad, Calif.) with addition of protease and phosphatase inhibitors. Tumor lysates were centrifuged to pellet insoluble debris, and the clarified supernatants were transferred to new tubes. pSmad2 was measured using a 6-Plex TGFbeta Signaling Magnetic Bead Kit (Millipore, Burlington, Mass.). All assays were carried out at room temperature. After blocking a 96-well black round-bottom plate with assay buffer for 10 minutes, 25 μL of the working microsphere bead mixture (beads were diluted to 1× with assay buffer from kit) and 25 μL of 1:10 diluted tumor lysate (1:10 dilution with assay buffer) were added to the plate. After overnight incubation at 4° C. with shaking, the bead mixtures were washed using a handheld magnetic separation block (EMD Millipore Catalog # 40-285). Beads with bound pSmad2 were incubated with 25 μL of biotinylated detection antibody solution for 1 hour, and then the bead mixtures were washed. For detection, 25 μL of streptavidin-PE solution was added and incubated for 15 minutes, and then 25 μL of amplification buffer was added with another incubation of 15 minutes. After washing, the beads were resuspended in 150 μL/well of sheath fluid (Bio-Rad catalog # 171-000055) and analyzed using a Bio-Plex 200 analyzer (Bio-Rad, Hercules Calif.). The mean fluorescence intensity (MFI) from each well was determined using Bio-Plex Manager Software, version 6.1 (Bio-Rad). The MFI minus the signal intensity of the blank well was used for further analysis.

Total Smad2 Bioassay: PathScan Total Smad2 Sandwich ELISA Kit (Cell signaling, Catolog #7244C) was used to determine the total Smad2 protein according to manufacturer's instructions. Tumor lysate samples were diluted 1:100 with diluent buffer, and 100 μL was added to the appropriate wells. The plate was incubated for 2 hours at 37° C. After washing the plate, detection solution (100 μL/well) was added, and the plate was incubated for 1 hour at 37° C. The plate was washed, and then 100 μL of HRP-linked secondary antibody was added and incubated for 30 minutes at 37° C. The plate was washed again, TM B substrate was added, and the plate was incubated for 30 minutes at room temperature. To quench the reaction, STOP solution was added to each well. The absorbance of the samples at 450 nm was measured on a Spectramax plate reader (Molecular Devices).

Phospho-Rb Ser807/811 Bioassay: The phospho-Rb protein S807/811 were analyzed in tumor lysates with a multiplex assay, which was developed and characterized using a 10-spot 96 well U-PLEX plate and unique linkers that were purchased from Meso-Scale Discovery (MSD). The phospho-Rb specific antibody, pS807/811 (8516BF) and total Rb antibody (9309BF) were purchased from Cell Signaling Technology (CST). In this 5-PLEX assay, the phospho-Rb specific antibody was biotinylated and coupled to U-PLEX Linkers. The linkers then self-assemble onto unique spots on the U-PLEX plate as the capture reagents. The properly diluted tumor lysates were added to the plate. After analytes in the sample bind to the capture reagents, the Rb detection antibody that was conjugated with electrochemiluminescent label (MSD GOLD SULFO-TAG) binds to the analytes to complete the sandwich immunoassay.

### Results

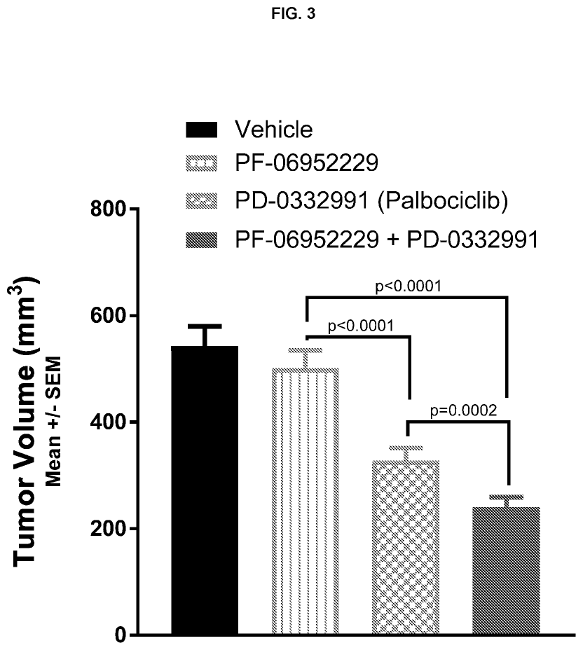

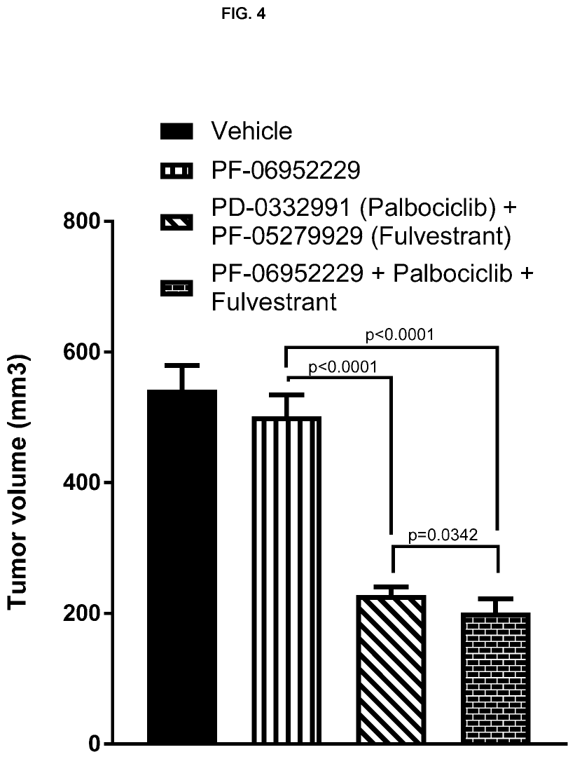

On Day 21 post-treatment initiation, tumor growth results show that treatment with the TGFβ inhibitor PF-06952229 monotherapy did not significantly inhibit tumor growth in the MCF7 xenograft tumor model; however, PF-06952229 treatment in combination with the CDK4/6 inhibitor palbociclib led to a significant combinatorial effect and thus an increase in tumor growth inhibition relative to PF-06952229 monotherapy (p <0.00001) and to palbociclib monotherapy (p=0.0002) (FIG. 3). When combined with palbociclib+fulvestrant, PF-06952229 also showed significant combinatorial effect, with a p=0.0342 when compared to palbociclib+fulvestrant treatment (FIG. 4).

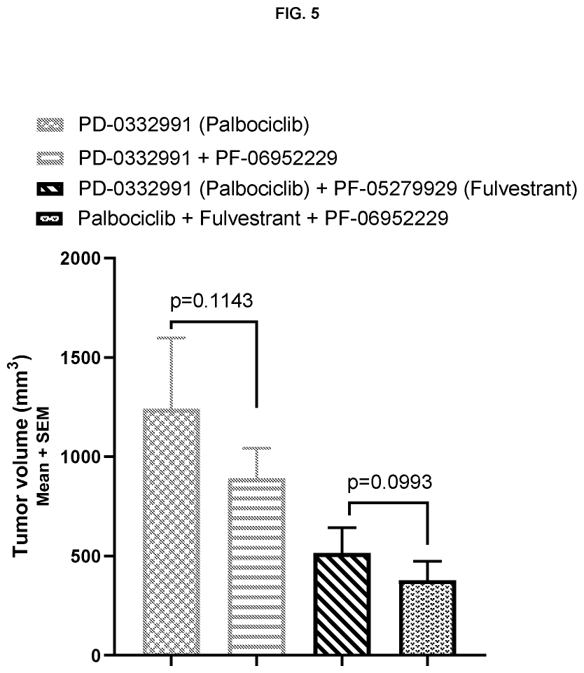

On the same day of the study (Day 21 post-treatment initiation), the animals in Group 2 (Palbociclib) were randomized to create two new treatment groups, with n=5 animals per group. TGFβ inhibitor PF-06592229 treatment was then added to one of the newly created groups, and palbociclib treatment continued for both newly created groups until Day 66 post treatment initiation, when the study ended. The same procedure was performed for Group 4 on Day 21, when animals in this group were randomized in two new treatment groups, and TGFβ inhibitor PF-06952229 treatment was added to one of these groups, while palbociclib+fulvestrant treatment continued for both newly created groups until Day 66. Although addition of TGFβ inhibitor PF-06952229 to palbociclib group or to palbociclib+fulvestrant groups did not have a statistically significant effect compared to palbociclib or palbociclib+fluvestrant alone, there was a trend for a greater tumor inhibition when TGFβ inhibitor PF-06952229 treatment was added to palbociclib or palbociclib+fluvestrant groups (FIG. 5).

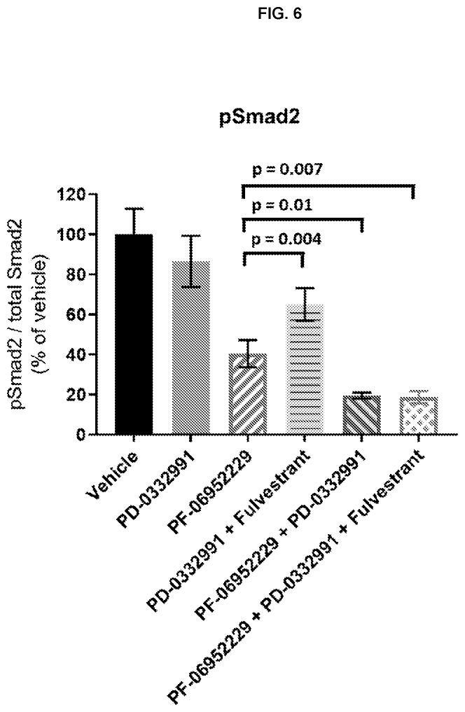

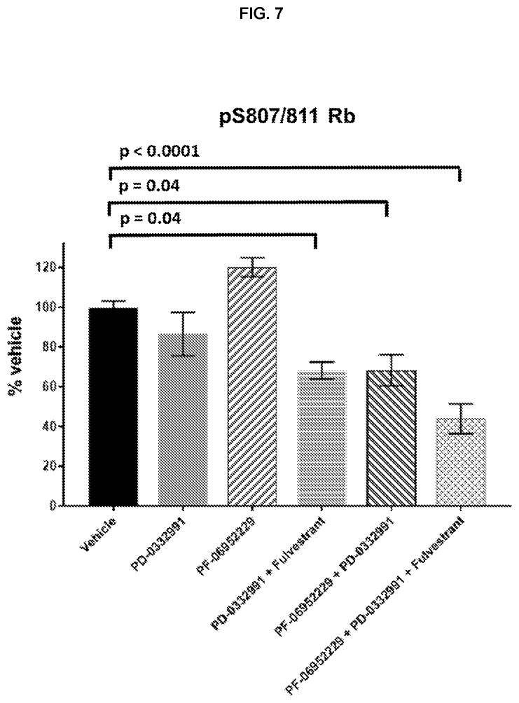

Biomarker analysis of tumor samples isolated on Day 21 post-treatment initiation demonstrated that treatment with TGFβ inhibitor PF-06592229 resulted in significant inhibition of pSMAD2, a key compoment of the TGFβ signaling pathway (FIG. 6). Modest inhibition of pSMAD2 was also observed in the palbociclib+fulvestrant group, however, the effect of TGFβ inhibitor PF-06952229 alone was superior to the palbociclib+fulvestrant combination (p=0.004) (FIG. 6). Strongest inhibition of pSMAD2 was observed in the groups where TGFβ inhibitor PF-06952229 was administered in combination with palbociclib or palbociclib+fulvestrant (˜80% inhibition in both groups), demonstrating the that addition of palbociclib improves the ability of PF-06952229 to downregulate pSMAD2 levels (p=0.01 and p=0.007, respectively) (FIG. 6). Phosphorylation of Rb is a downstream biomarker of CDK4/6 inhibition in cancer cells. Treatment with single agent palbociclib resulted in slight decrease in pS807/811 Rb levels on Day 21, while single agent treatment wth TGFβ inhibitor PF-06952229 resulted in a slight increase in these same phospo-proteins (FIG. 7). Improved inhibition of pS807/811 Rb levels was observed with the combination of palbociclib and fulvestrant (p=0.04), and a similar improvement was observed in tumors treated with the combination of palbociclib and PF-06952229 (p=0.04). The addition of TGFβ inhibitor PF-06952229 to the combination of palbociclib+fulvestrant resulted in the strongest inhibition of pS807/811 Rb levels (p<0.0001) (FIG. 7). Overall, the data indicates that there is a trend toward improved inhibition of pS808/811 Rb when TGFβ inhibitor PF-06952229 is used in combination with palbociclib alone or palbociclib+fulvestrant.

### Conclusions

TGFβ inhibitor PF-06952229 combination with the CDK4/6 inhibitor palbociclib or with palbociclib plus fulvestrant, a selective estrogen receptor degrader, led to greater tumor growth inhibition relative to PF-06952229 or palbociclib monotherapies, or to palbociclib+fulvestrant combination, in the MCF-7 ER⁺ HER2⁻ xenograft breast cancer tumor model. Addition of the TGFβ inhibitor PF-06952229 to animals previously receiving CDK4/6 inhibitor palbociclib or palbociclib+fulvestrant treatment for 21 days led to a trend to increased tumor growth inhibition relative to palbociclib monotherapy or to palbociclib+fulvestrant combination. Moreover, the combination of TGFβ inhibitor PF-06952229 +palbociclib or palbociclib+fulvestrant resulted in increased inhibition of downstream signaling pathways for both the TGFβR1 (pSMAD2) and CDK4/6 (pS807/811 Rb).

1-28. (canceled)

29. A method for treating cancer comprising administering to a patient in need thereof; an amount of a TGFβ inhibitor in combination with an amount of:
a. a CDK4/6 inhibitor; or
b. a CDK2/4/6 inhibitor;
wherein the amounts together are effective in treating cancer.

30. The method of claim 29, wherein the TGFβ inhibitor is 4-(2-(5-chloro-2-fluorophenyl)-5-isopropylpyridin-4-ylamino)-N-(1,3-dihydroxypropan-2-yl)nicotinamide or a pharmaceutically acceptable salt thereof.

31. The method of claim 29, wherein the CDK4/6 inhibitor is palbociclib, or a pharmaceutically acceptable salt thereof.

32. The method of claim 29, wherein the CDK2/4/6 is 6-(difluoromethyl)-8((1R,2R)-2-hydroxy-2-methylcyclopentyl)-2-(1-(methylsulfonyl)piperidin-4-ylamino)pyrido[2,3-d]pyrimidin-7(8H)-one, or a pharmaceutically acceptable salt thereof.

33. The method of claim 29, wherein the cancer is breast cancer.

34. The method of claim 33, wherein the breast cancer is hormone receptor positive (HR+) or human epidermal growth factor receptor 2 negative (HER2−) breast cancer.

35. The method of claim 33, wherein the breast cancer is advanced breast cancer.

36. The method of claim 33, wherein the breast cancer is metastatic breast cancer.

37. The method of claim 29, wherein the cancer is colon cancer.

38. A method for treating cancer comprising administering to a patient in need thereof an amount 4-(2-(5-chloro-2-fluorophenyl)-5-isopropylpyridin-4-ylamino)-N-(1,3-dihydroxypropan-2-yl)nicotinamide or a pharmaceutically acceptable salt thereof; and an amount of (a) palbociclib, or a pharmaceutically acceptable salt thereof; or (b) 6-(difluoromethyl)-8-((1R,2R)-2-hydroxy-2-methylcyclopentyl)-2-(1-(methylsulfonyl)piperidin-4-ylamino)pyrido[2,3-d]pyrimidin-7(8H)-one, or a pharmaceutically acceptable salt thereof.

39. The method of claim 38, wherein the cancer is breast cancer.

40. The method of claim 38, wherein the cancer is colon cancer.

41. The method of claim 38, said method further comprising administering an amount of fulvestrant.

42. A combination of 4-(2-(5-chloro-2-fluorophenyl)-5-isopropylpyridin-4-ylamino)-N-(1,3-dihydroxypropan-2-yl)nicotinamide, or a pharmaceutically acceptable salt thereof; and palbociclib, or a pharmaceutically acceptable salt thereof; for use in the treatment of breast cancer or colon cancer.

43. The combination of claim 42, further comprising fulvestrant.

44. A combination of 4-(2-(5-chloro-2-fluorophenyl)-5-isopropylpyridin-4-ylamino)-N-(1,3-dihydroxypropan-2-yl)nicotinamide, or a pharmaceutically acceptable salt thereof; and 6-(difluoromethyl)-8-((1 R,2R)-2-hydroxy-2-methylcyclopentyl)-2-(1-(methylsulfonyl)piperidin-4-ylamino)pyrido[2,3-d]pyrimidin-7(8H)-one, or a pharmaceutically acceptable salt thereof; for use in the treatment of breast cancer or colon cancer.

45. The combination of claim 44, further comprising fulvestrant.
